(19) United States
(12) Patent Application Publication
(10) Pub. No.: US 2008/0269107 A1
(43) Pub. Date: Oct. 30, 2008

(54) Methods and Compositions for Treatment of Cancers with Hoxb4

(76) Inventor: Kamal K. Mubarak, Gainesville, FL (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/090,750

(22) PCT Filed: Oct. 23, 2006
(86) PCT No.: PCT/US2006/041579

§ 371 Date: Apr. 18, 2008

Related U.S. Application Data
(60) Provisional application No. 60/729,277, filed on Oct. 21, 2005.

(51) Int. Cl. A61K 38/17; A61P 35/00

(52) U.S. Cl. 514/2

(57) ABSTRACT

In some embodiments, without limitation, the invention comprises methods and compositions for the treatment of mammalian cancers, comprising the administration of HOXB4 protein and/or the overexpression of HOXB4 nucleotide sequence in order to induce apoptosis or otherwise decrease the survival of the target cancer cells.

### CROSS-REFERENCE TO RELATED APPLICATIONS

This application claims priority based on U.S. Provisional Patent Application No. 60/729,277, filed Oct. 21, 2005, which is hereby incorporated by reference in full.

### FIELD OF THE INVENTION

The invention relates generally to the field of cancer therapeutics and treatments, including without limitation, methods and compositions for cancer treatments comprising novel therapeutic molecules.

### BACKGROUND

As the understanding of the etiology of cancers expands, researchers continue to look for ways to improve corresponding survival rates of human patients with malignancies. Conventional treatments, such as chemotherapeutic regimes, have increased response rates and improved survival, but are most effective only when the tumor cells proliferate faster than normal cells, which is not always true. Increasingly, cancer research has focused on understanding molecular pathways of cancer transformation and progression in order to develop therapies to improve cure rates while reducing nonspecific cytotoxicity that may affect normal cells. Part of the corresponding thought is to develop novel molecular targets in such pathways in order to provide additional therapeutic options. While cancer research has provided additional understanding of processes responsible for cancer growth and has identified numerous molecular targets for cancer therapy, an unmet need remains for therapeutic options that are able to utilize molecular pathways in order to prevent, reduce, or eliminate cancers in a patient.

### SUMMARY OF THE INVENTION

Without limiting the embodiments to only those described in this section, the invention comprises methods and compositions for the treatment of cancers using HOXB4 protein and/or the overexpression of HOXB4 nucleotide sequence. Embodiments of the invention comprise novel tools for the treatment of cancers.

We have discovered unexpectedly that when HOXB4 is overexpressed in mammalian cancer cells, or when such cells are exposed to HOXB4 protein in therapeutically effective amounts, their survival is decreased. Our findings suggest that one mechanism for this decrease is the unexpected induction of apoptosis in the cancer cells by HOXB4. In accordance with some embodiments, without limitation, HOXB4 induces apoptosis, whether by transfection of the HOXB4 gene or by transmembrane delivery of HOXB4 protein. Our findings indicate for the first time that HOXB4 is a critical factor in decreasing cancer cell survival, and our discovery presents new therapeutic options for cancers.

Other aspects of the invention will be apparent to those skilled in the art after reviewing the drawings and the detailed description below.

### BRIEF DESCRIPTION OF DRAWINGS

The present invention will now be described, by way of example, with reference to the accompanying drawings, in which:

FIG. 1 is a chart of a cell growth curve of REH cells transfected with pLNCX2-IRES-GFP control plasmid and pLNCX2-HOXB4-IRES-GFP construct.

FIG. 2 are pictures showing the immunocytochemistry with HOXB4 antibody 2 weeks after transfection.

FIG. 3 are pictures showing the results of a TUNEL assay 4 weeks after transfection.

FIG. 4 is a picture showing caspase-3 expression 4 weeks after transfection, caspase-8 expression 4 weeks after transfection, and FLASH expression 3 weeks after transfection.

FIG. 5 is a picture showing caspase-9 expression 4 weeks after transfection and SMAC expression 3 weeks after transfection.

FIG. 6 is a chart showing cell death in WSU-DLCL2 cells with HOXB4 over-expression.

FIGS. 7(A)-(D) are charts showing dose-response findings concerning the administration of HOXB4 to different cell types.

FIG. 8 is a chart showing a possible mechanism of HOXB4-induced apoptosis in REH cells.

FIG. 9 is a chart of a cell growth curve of REH cells transfected with pLNCX2 control plasmid and pLNCX2-HOXB4-IRES-GFP construct.

FIG. 10 are pictures showing immunocytochemistry with HOXB4 antibody 2 weeks after transfection.

FIG. 11 are pictures showing the results of a TUNEL assay after 4 weeks of transfection.

FIG. 12 are pictures showing caspase-3 expression after 4 weeks of transfection.

FIG. 13 is a chart showing FLASH RNA expression in HOXB4 transfected REH cells and controls.

FIG. 14 are pictures showing FLASH expression after 3 weeks of transfection.

FIG. 15 are pictures showing caspase-9 expression 4 weeks after transfection and SMAC expression 3 weeks after transfection.

FIG. 16 is a diagram showing a B cell differentiation pathway and malignancies that arise from different stages.

FIG. 17 is a chart showing growth inhibition of hematopoietic malignant cell lines with HOXB4 over-expression.

FIG. 18 are a picture and charts showing production of HOXB4 protein and addition of protein to culture of REH and DLCL2 cells.

FIG. 19 is a chart of DLCL2 cell growth after treatment with beta-galactosidase and HOXB4.

FIG. 20 is a chart of REH cell growth after treatment with beta-galactosidase and HOXB4.

FIG. 21 is a chart of ER-293-HOXB4 cell lines with and without ponasterone A in culture medium.

FIG. 22 is a chart of ER-293-HOXB4 #6 cell line with 5-azacytidine and trichostatin A.

FIG. 23 is a Western blot of apoptosis mediators.

FIG. 24 is a Western blot of HOXB4 protein.

FIG. 25 is a chart of cell growth of REH cells with HOXB4 protein with and without caspase-8 inhibitors.

### DETAILED DESCRIPTION

In some embodiments, without limitation, the invention comprises methods and compositions to induce apoptosis in cancer cells in mammalian subjects, including humans, by effecting the overexpression of HOXB4 in those cells. Some embodiments of the invention also comprise the administration of HOXB4 protein in a therapeutically effective amount in order to induce apoptosis and/or otherwise decrease survival in target cancer cells. Other embodiments include, without limitation, the induction of overexpression of the HOXB4 gene, for example, by activation of the gene in targeted cancer cells in vivo, or by activation of the HOXB4 gene in vivo after administration of the gene via transfection. Thus, embodiments of the invention comprise novel compositions and tools for the treatment of cancers.

We have discovered unexpectedly that HOXB4, when overexpressed in hematopoietic and other cancer cell lines, causes apoptosis. The cells progressively decline in number, express caspase-3, and show DNA fragmentation, all features of apoptosis. Overexpression of HOXB4 in pre-B-ALL REH cells in vitro activates a pathway that leads to cell death via apoptosis. We have shown that HOXB4 can passively translocate across cell membranes and thereby induce apoptosis when added to cell culture of B-cell malignancies. HOXB4 can exert these effects in vivo also when injected into tumors, causing reduction in size. As one example only, when HOXB4 protein is injected into DLCL2 tumors in SCID mice, these tumors are reduced in size. Therefore, HOXB4 is a useful cancer therapeutic agent.

We have shown unexpectedly that activation of HOXB4 and downstream genes may act a useful chemotherapeutic strategy for hematopoietic and other malignancies without causing immunosuppression. Conversely, used in combination with established protocols, such therapy may enhance immunity during cytotoxic therapy without interfering with antineoplastic effects.

In accordance with some embodiments, without limitation, HOXB4 can be used and administered in any pharmaceutically acceptable form and manner. Consistent with our findings, administration of HOXB4 can induce apoptosis in cancer cells. Therefore, by administering such a composition to a patient, it is possible to induce apoptosis in cancer cells, and a pharmaceutical composition comprising the HOXB4 protein or other compound that results in HOXB4 overexpression in the cancer cell, and a method comprising a step of administering to a patient an effective amount of same, can be applied in the treatment and prophylaxis of cancer.

Some embodiments of the invention comprise, without limitation, methods and compositions to induce apoptosis in mammalian cancer cells, including without limitation, in REH, WSU-DLCL2, WSU-FSCCL, and WSU-WM cells, by transmembrane delivery of HOXB4 protein. Embodiments of the invention also comprise, without limitation, methods, compositions, and systems to overexpress HOXB4 in order to induce apoptosis in mammalian subjects having cancer cells in their bodies.

Some embodiments comprise a method of decreasing the survival of cancer cells in a mammal, including the step of administering to the mammal a compound comprising HOXB4 protein in a therapeutically effective amount that decreases the survival of at least some of the cancer cells. Embodiments also include, without limitation, a method of inducing apoptosis in cancer cells in a mammal, comprising the step of administering to the mammal a compound comprising HOXB4 protein in a therapeutically effective amount that induces apoptosis in at least some of the cancer cells. The HOXB4 protein may comprise a polypeptide of all or part of SEQ ID NO. 1.

Without limitation, some embodiments comprise a method of decreasing the survival of cancer cells in a mammal, comprising the step of transfecting the mammal with a HOXB4 nucleotide sequence in a therapeutically effective amount that decreases the survival of at least some of the cancer cells. Embodiments also comprise, without limitations, a method of inducing apoptosis in cancer cells in a mammal, comprising the step of transfecting the mammal with a HOXB4 nucleotide sequence in a therapeutically effective amount that induces apoptosis in at least some of the cancer cells. The HOXB4 nucleotide sequence may comprise all or part of SEQ ID NO. 2.

Medicaments, pharmaceutical compositions, and uses of same to decrease survival of cancer cells in a mammal are also disclosed. These may include, without limitation, a HOXB4 protein comprising a polypeptide of all or part of SEQ ID NO. 1, and/or a HOXB4 nucleotide sequence comprised of all or part of SEQ ID NO. 2.

Without limiting other possible modes of administration, introduction of HOXB4 nucleic acid may be accomplished by recombinant viral infection. Higher efficiency can be obtained due to virus' infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

The recombinant vector can be administered in several ways. For example, the procedure can take advantage of the target specificity of viral vectors and consequently do not have to be administered locally at the diseased site. However, local administration can provide a quicker and more effective treatment. Administration can also be performed by, for example, intravenous or subcutaneous injection into the subject. Following injection, the viral vectors will circulate until they recognize host cells with the appropriate target specificity for infection.

An alternate mode of administration can be by direct inoculation of HOXB4 protein locally at the site of the disease or pathological condition or by inoculation into the vascular system supplying the site with nutrients. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the HOXB4 nucleotide sequence and/or non-viral vectors described above into recipient cells within the inoculated area. Such transfection vehicles are known by one skilled within the art.

In accordance with some embodiments of the invention, HOXB4-related compounds are administered and dosed in accordance with good medical practice, taking into account the clinical condition of the individual patient, the site and method of administration, scheduling of administration, patient age, sex, body weight and other factors known to medical practitioners. The pharmaceutically or therapeutically “effective amount” for purposes herein is thus determined by such considerations as are known in the art. The amount must be effective to achieve improvement, including but not limited to, improved survival rate or more rapid recovery of the patient, decrease in survival of cancer cells, or improvement or elimination of symptoms and other indicators as are selected as appropriate measures by those skilled in the art.

In the method of the present invention, the compound of the present invention can be administered in various ways and is not limited to a particular way. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intranasal, transdermal or transmucosal administration, as well as intrathecal and infusion techniques. Implants of the compounds may also be also useful.

The patient being treated is a warm-blooded animal and, in particular, mammals including humans. Pharmaceutically acceptable carriers, diluents, adjuvants and vehicles as well as implant carriers generally refer to inert, non-toxic solid or liquid fillers, diluents or encapsulating material not reacting with the active ingredients of the invention. The compound may be appropriately formulated into conventional preparations (tablets, granules, capsules, powders, inhalants, etc.) with an inert carrier depending on the administration route and used.

It is noted that humans are treated generally longer than the mice or other experimental animals exemplified herein which treatment has a length proportional to the length of the disease process and drug effectiveness. The doses may be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated.

The following examples of some embodiments of the invention are provided without limiting the invention to only those embodiments described herein.

### EXAMPLE 1

Homeobox (“HOX”) proteins are transcriptional regulators that control the expression of batteries of downstream effector genes. HOX proteins define the segmental body plan, control differentiation, and regulate major metabolic pathways, among other functions.

Acting in concert with trithorax and Polycomb proteins, these “master control genes” regulate the organization of the body plan, control major metabolic pathways, initiate developmental programs, and cause apoptosis in certain developing cell types. There are 39 “clustered” or class I homeobox protein encoding genes, which are found arranged sequentially on chromosomes 2, 7, 12, and 17. These clusters arose by duplication during evolution.

The precise interactions of homeobox proteins within themselves and with others have not been fully elucidated. It has been proposed that homeobox, trithorax, and Polycomb proteins function as a “self-regulating network” of transcriptional regulators that regulate each others' expression at the transcription level, and can modify the chromatin structure, repressing or expressing large segments of the genome.

Homeobox genes have been referred to as “master control genes” because turning on a single homeobox gene can cause the unfolding of a complex genetic program. For example, the Aniridia gene induces the development of a complete eye by initiating the complex coordination of thousands of genes in numerous cell types. The Aniridia gene is conserved from Drosophila to humans. In fact, the Aniridia gene taken from humans and overexpressed in any segment of the developing body of Drosophila will cause the development of an insect eye in that part of the body. Thus a conserved human gene is able to orchestrate myriad insect genes that form the multi-lens insect eye.

Conserved homeobox genes can also produce homeotic transformation, with effects that occur only at a critical time in embryogenesis. Expressed too early or too late, the gene is unable to induce the formation of an eye. This time- and stage-sensitive effect of homeobox gene expression may be explained by the interaction of one gene with other members of this family. Presumably homeobox genes do not act alone in initiating an entire cascade of gene expression leading to organ development. It therefore follows that Aniridia interacts with other genes that are expressed at that time to achieve its downstream effects. It is quite possible that the effect of a given homeobox protein is determined by the state of the homeobox-Polycomb-trithorax “network”, which in turn determines cell type. Therefore a given homeobox protein may have different effects in different cell types.

Every cell type in the human body arises from a single embryonic stem cell via a process of cell division and differentiation, but almost all cells in a higher organism have identical DNA. Yet there are over 200 cell phenotypes in a typical mammal. How cells manage to remember their own cell type, transmit this information to their progeny despite going through mitosis, and then have their progeny differentiate their phenotype in an orderly manner, all the while maintaining the same DNA, is not understood very well. It is thought that the cellular memory of phenotype is not encoded in the DNA, but is epigenetic.

Integral to any process of growth and differentiation is apoptosis. Those cells that are no longer needed are fated to die, and have to be removed without causing an inflammatory reaction. Homeobox, Polycomb, and trithorax proteins orchestrate this process of cell memory, cell identity, and cell destiny. These processes may be collectively called cell type control.

The fact that these proteins play an important role in development is well established. In the hematopoietic system, the homeobox proteins are intimately involved in differentiation, and overexpression of some members can lead to malignant transformation.

Arguably, cancer can be described as a genetic abnormality where there is dysregulation of cell type control. This dysregulation leads to transformation of a benign cell into a malignant and proliferative cell type. There are clonal populations within any tumor that escape selective pressures and progress along a pathway that leads to metastases. It is now being recognized that homeobox genes may orchestrate these processes in cancer, in particular, hematological malignancies. Expression of some homeobox proteins is abnormal in many primary tumors. In fact, as the tumor progresses into metastases, there is further misexpression and downregulation of homeobox genes correlating with cancer stage and suggesting that homeobox proteins have a role in tumor suppression. These developments have been recently reviewed.

Several homeobox genes are implicated in apoptosis of certain cell types. Targeted disruption of HOXB1 induces apoptosis in rhombomere 4 in the mouse via sonic hedgehog and MASH1. Similarly, HOX11 (a divergent homeodomain protein) prevents apoptosis in the spleen in a mouse model. Interruption of HOXA9 causes mice to develop small thymus glands and induces apoptosis in thymocytes.

The best-known example of apoptosis caused by a homeobox gene is in the central nervous system. Morsi El-Kadi and colleagues have demonstrated that HOXB4 directly regulates caspase-8 associated protein 2 (FLASH) in the notochord of Xenopus during development. FLASH binds to the death effector domain of caspase-8, a component of the tumor necrosis factor (TNF)-mediated (extrinsic) apoptosis pathway. This leads to the involution of the notochord, a critical step in embryogenesis.

A link between homeobox proteins and cell cycle has been postulated. However, very few homeodomain proteins have been shown to regulate, or be regulated by, the cell cycle. Overexpression of HOX11 in Xenopus oocytes as well as in mammalian cells releases G2 arrest. The HOX11 protein is expressed at the highest level at the G1/S boundary and probably functions as a transcription factor for G1 progression in T cell acute lymphoblastic leukemia. Overexpression of the diverged homeodomain protein HSIX abolishes the G2 checkpoint in MCF7 breast cancer cells in response to radiation. The diverged GAX/MEOX2 protein is markedly downregulated in vascular smooth muscle cells in response to mitogens. Its levels rise subsequently, indicating that it has a role in G0/G1 transition. These findings have been recently reviewed.

One member of the HOX protein family, HOXB4, causes ex vivo clonal expansion of hematopoietic stem cells. Therefore HOXB4 has been extensively studied as an agent that can augment autologous bone marrow transplantation.

By comparison, even though cancer cells have self-renewing potential similar to stem cells, their biological behavior is completely different. They seem to have lost the capacity to stop proliferating, or to undergo apoptosis, in response to physiologic stimuli. Stem cells retain the ability to differentiate into several terminally differentiated cell types, but cancers typically do not, unless subjected to differentiation therapy. Clearly, the cellular memory, identity, and destiny are different for these two types of cells, despite the fact that they share the potential for limitless growth. If indeed the “network” of homeobox, trithorax, and Polycomb proteins is responsible for this difference, then the overexpression of a member of this “network” may have the divergent effects in cancer and stem cells. We have demonstrated such a dissonant effect with our studies, with concomitant its therapeutic potential.

The role of homeobox, trithorax, and Polycomb protein families has been difficult to study. These are some of the most ancient genes in the eukaryotic genome and are highly conserved across species. At the genetic level, all homeobox genes share a conserved 183-nucleotide sequence, called the homeobox, which encodes a 61-amino acid sequence called the homeodomain. At the protein level, there is very little, if any, difference in the amino acid sequence among mammals. Thus raising antibodies has proven very difficult.

Given the key role of these proteins in cell type control, we determined that RNA expression profiling of these three families of genes would be a method of choice to specifically detect expression of each gene product. To examine the role of all 80 members of the homeobox, trithorax, and Polycomb gene families, a targeted microarray was developed, while recognizing that all homeobox genes share a 183-nucleotide sequence (the “hoxbox”).

We constructed a low-density, custom-targeted microarray of homeobox, trithorax, and Polycomb gene families. Because homeobox, trithorax, and Polycomb genes have a well-defined role in differentiation as well as cancer, we hypothesized that several members of these gene families would show differential expression if hematopoietic malignancies were to be subjected to differentiation therapy. Therefore we used a targeted microarray to study an experimental model of differentiation therapy. Bryostatin-1 is a macrocyclic lactone that has been extracted and purified from the marine bryozoan Bugula neritina. It is a protein kinase C modulator similar to the 12-O-tetradecanoyl phorbol 13-acetate (TPA), but is unique in that bryostatin-1 lacks tumor promoting activity. Other researchers have worked extensively with leukemia and lymphoma cell lines and have an experimental model using bryostatin-1 in REH cells, a pre-B-ALL cell line. Once exposed to bryostatin-1, the REH cell line differentiates along a B-lineage pathway into a monocytoid B-lymphocyte in vitro as well as in vivo. There is increasing CD11c and CD22 co-expression with decreasing CD10 and CD19 expression as this differentiation progresses. These cell surface markers can be used to follow the progress of differentiation.

To determine which genes of these 3 families were differentially regulated in bryostatin-1-mediated cell type control, RNA from bryostatin-1-treated REH cells was isolated and expression profiling was performed using untreated REH cells as controls. Bryostatin-1 suppressed the expression of HOXB4 RNA along with several chromobox genes of the Polycomb family (data not shown).

The role of HOXB4 has not been studied in REH cells before. However, when HOXB4 is overexpressed in embryonic stem cells, it causes these cells to differentiate into hematopoietic stem cells (HSCs). When HOXB4 is highly overexpressed in HSCs there is clonal expansion of HSCs, preventing their differentiation into more terminal cell types. Lower levels of HOXB4 allow myeloid differentiation of HSCs. In a mouse model of bone marrow transplantation, HOXB4-transduced HSCs have been shown to fully reconstitute the HSC compartment without overriding the regulatory mechanisms that maintain the HSC pool size within normal limits. Fascinatingly, when followed for up to 1 year, none of the mice showed any leukemic transformation despite the pro-proliferative effects of HOXB4.

HOXB4 has also been shown to have a completely different effect. In the notochord of the Xenopus, HOXB4 directly regulates the expression of caspase-8 associated protein 2, FLASH, (Casp8ap2, FLICE associated huge protein) in the notochord of Xenopus during development. FLASH binds to the death effector domain (DED) of caspase-8, a component of the tumor necrosis factor (TNF)-mediated (extrinsic) apoptosis pathway. This leads to activation of caspase-8 that causes apoptosis in the degenerating notochord.

Since hematological malignancies have a similar phenotype to hematopoietic stem cells, we expected that HOXB4 would have similar effects in both cell types. Therefore we hypothesized that suppression of HOXB4 RNA by bryostatin-1 was the cause of the differentiating effect seen in this therapeutic model.

We hypothesized that several members of the HOX gene family would show differential expression if hematopoietic malignancies were subjected to differentiation therapy. To test our hypothesis that downregulation of HOXB4 RNA results in differentiation of REH cells, we overexpressed HOXB4 in REH cells, expecting that it would cause expansion of REH cells, similar to hematopoietic stem cells. This overexpression could be a cause of resistance to bryostatin-1 therapy.

We therefore studied an experimental model using a differentiating agent, bryostatin-1, on a pre-B acute lymphoblastic leukemia (ALL) cell line, REH. Using a microarray, we found that bryostatin-1 suppressed the expression of HOXB4 RNA (data not shown). We hypothesized that suppression of HOXB4 RNA by bryostatin-1 was the cause of the differentiating effect seen in the bryostatin-1 therapeutic model. Therefore overexpression should be pro-proliferative and should cause resistance to differentiating agents. We directly tested our hypothesis as follows.

Cell Culture: REH and WSU-DLCL2 cell lines are derived from pre-B acute lymphoblastic leukemia and diffuse large cell lymphoma respectively. These cell lines were routinely cultured in suspension using RPMI 1640 medium with L-glutamine and 10% heat-inactivated fetal calf serum at 37° C. and 5% CO₂ in 25 cm² flasks. Medium was changed every 3 to 7 days, depending on growth rate. G418 (Promega, Madison, Wis.) was used for selection of neomycin resistance at a concentration of 1000 mg/L for REH cells and 400 mg/L for WSU-DLCL2.

Plasmid Transfection: The pLNCX2-HOXB4, pLNCX2-HOXB4-IRES-GFP, pLNCX2-IRES-GFP and pLNCX2 (control plasmid) (Clontech, Mountain View, Calif.) were transfected into REH and WSU-DLCL2 cell lines using Lipofectamine Plus (Invitrogen, Carlsbad, Calif.) according to the manufacturer's protocol. Neomycin selective pressure was applied by adding G418 to the culture after 3 days. Cells were cultured as above. The REH cell line typically stabilizes 3 weeks after addition of G418 and then enters an exponential growth phase after 4 weeks. The WSU-DLCL2 cell line follows a similar course.

Immunocytochemistry: Using a Cytospin centrifuge (Thermo Electron, Waltham, Mass.), REH cells in suspension were deposited on a glass slide. Cells were fixed with 4% methanol-free formaldehyde and then washed with phosphate buffered saline (PBS) for 5 minutes. The slide was incubated with the primary antibody for one hour at room temperature and then left at 4° C. overnight. The slide was then washed with PBS 3 times for 5 minutes before incubation with the secondary biotinylated antibody for 3 hours at room temperature. After another wash cycle, the Vectastain ABC kit (Vector Labs, Burlingame, Calif.) was used to stain the cells according to the manufacturer's protocol. The slides were visualized with Olympus (Tokyo, Japan) microscope BX-40 at a magnification of 100× and photographed using a Photometrics CoolSnap cf digital camera (Roper Scientific, Tucson, Ariz.) connected to a computer running MCID Elite 7.0 software (Imaging Research, St Catherine, ON).

The primary antibodies used included the rat monoclonal HOXB4 antibody (Developmental Studies Hybridoma Bank, University of Iowa), anti-caspase-3 (activated) antibody (Santa Cruz Biotechnology, Santa Cruz, Calif.), anti-caspase-8 (activated) antibody (Leinco, St Louis, Mo.), anti-caspase-9 (activated) antibody (Abcam, Cambridge, Mass.), anti-SMAC antibody (Abcam, Cambridge, Mass.), and anti-FLASH antibody (Imgenex, San Diego, Calif.).

TUNEL Assay: Cells were attached to a glass microscope slide by Cytospin centrifuge as above. The slide was immersed in 4% methanol-free formaldehyde for fixation. The DeadEnd Fluorometric TUNEL System (Promega, Madison, Wis.) was used to label the cells. The slides were visualized under an Olympus microscope BX-40 at a magnification of 400× and photographed using a Photometrics CoolSnap cf digital camera connected to a computer running MCID Elite 7.0 software.

Our results showed unexpectedly that overexpression of HOXB4 causes REH cell death. The REH cells were transfected with the pLNCX2-HOXB4-IRES-GFP and pLNCX2-IRES-GFP control vector. After a typical transfection, it takes approximately 4 weeks for REH cells to recover and begin exponential growth. As expected, the REH cells transfected with pLNCX2-IRES-GFP began to grow and entered exponential phase after 4 weeks. The REH cells transfected with pLNCX2-HOXB4-IRES-GFP followed the same quiescent path as the pLNCX2-IRES-GFP transfected cells for 4 weeks, but instead of entering an exponential phase, inexplicably died.

Repeating the experiment produced the same result. Deleting the IRES-GFP segment (pLNCX2-HOXB4) had no effect. The cells died again. Using the native pLNCX2 vector abrogated the effect-the cells behaved just like the pLNCX2-IRES-GFP controls, and survived. After a total of 5 attempts that confirmed the same result, this effect was investigated further.

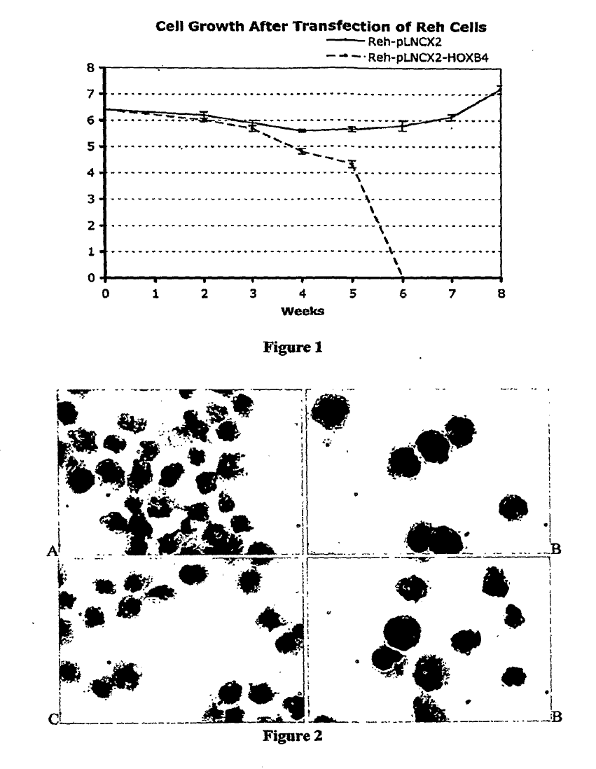

To quantify the phenomenon of cell death, the transfected cells were repeatedly sampled at weekly intervals and counted. Logarithms (base 10) of cell counts were obtained and plotted. FIG. 1 shows the mean of 3 such experiments with standard deviations. FIG. 1 shows REH cells transfected with pLNCX2-IRES-GFP control plasmid and pLNCX2-HOXB4-IRES-GFP construct. The cell counts remained close to each other for about 3 or 4 weeks and then began to diverge. As the control cells entered the exponential phase, the HOXB4 cells died off. Results with pLNCX2-HOXB4 plasmid without the IRES-GFP were similar to the pLNCX2-HOXB4-IRES-GFP plasmid and results of pLNCX2 were similar to pLNCX2-IRES-GFP (data not shown). The curves began to diverge once the cells reach the exponential stage after transfection at 4 weeks. Control cells grow normally, but HOXB4 transfectants die off rapidly. Repeated experiments showed similar results.

To determine if this effect of cell death was attributable to HOXB4 protein expression, the cells transfected with HOXB4 gene were examined under an Olympus microscope BX-40 using fluorescent filters. The transfected cells were fluorescent, indicating that green fluorescent protein (GFP) was being expressed via the internal ribosome entry site (IRES) (data not shown).

A HOXB4 rat monoclonal antibody (Developmental Studies Hybridoma Bank, University of Iowa) was obtained and immunocytochemistry was performed on Cytospin specimens of REH cells 2 weeks after transfection (FIG. 2). FIG. 2 shows immunocytochemistry with HOXB4 antibody 2 weeks after transfection. The top left panel A shows REH cells transfected with control plasmid pLNCX2-IRES-GFP and stained with anti-HOXB4 antibody. The top right panel B shows REH cells transfected with pLNCX2-HOXB4-IRES-GFP plasmid and stained similarly. The bottom left panel C shows REH cells transfected with pLNCX2-HOXB4-IRES-GFP plasmid and stained with rat IgG (negative control). The bottom right panel D shows REH cells transfected with pLNCX2-HOXB4-IRES-GFP plasmid stained with anti-CD45RO (leukocyte common antigen) antibody (positive control). REH cells transfected with control pLNCX2-IRES-GFP plasmid showed similar results with both control antibodies (micrographs not shown). As expected, the REH cells showed staining with the anti-HOXB4 monoclonal antibody 2 weeks after transfection with pLNCX2-HOXB4-IRES-GFP. The cells transfected with pLNCX2-IRES-GFP had no HOXB4 staining. An anti-CD45 antibody (Sigma, St Louis, Mo.) was used as positive control. Similar results were obtained at 3 and 4 weeks after transfection.

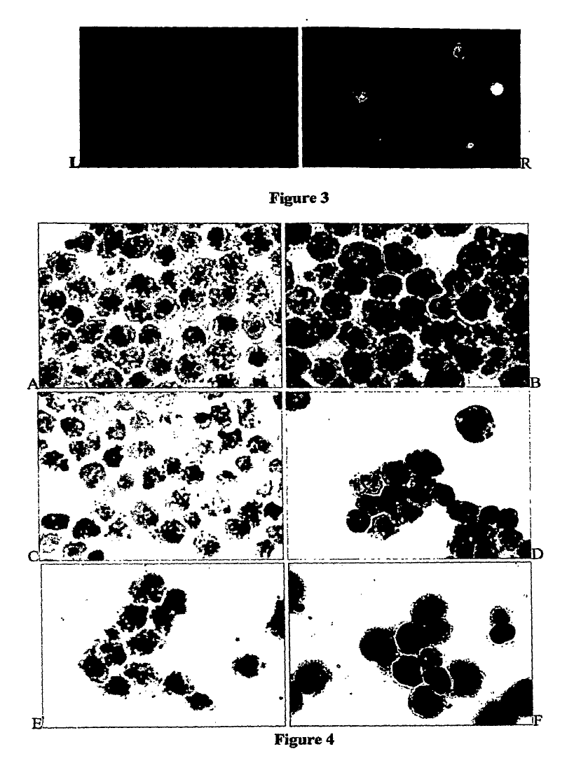

Our results also showed that REH cells overexpressing HOXB4 undergo DNA disintegration. To investigate whether the observed cell death was due to apoptosis, a TdT-mediated dUTP nick-end labeling (TUNEL) assay was performed to detect DNA fragmentation. For this series of experiments, the IRES-GFP segment was removed from both plasmids to prevent interference with fluorescence detection. After 4 weeks, the REH cells transfected with pLNCX2-HOXB4 had DNA fragmentation, a feature of apoptosis, in contrast to control cells that had no DNA fragmentation (See FIG. 3). FIG. 3 shows a TUNEL assay 4 weeks after transfection. REH cells with control pLNCX2 plasmid (left) demonstrated no DNA fragmentation (red fluorescence). In contrast, the REH cells with pLNCX2-HOXB4 plasmid (right) demonstrated cell death (green) and DNA fragmentation (yellow). DNA fragmentation could be demonstrated faintly as early as 3 weeks but not prior to this at 2 weeks (data not shown).

Our results also showed that REH cells overexpressing HOXB4 also express increased caspase-3, caspases-8, and FLASH. To confirm that enzymes that mediate apoptosis were present, a caspase-3 assay was performed. Apoptosis is a carefully orchestrated, active, metabolic process that is mediated via aspartate-specific cysteine proteases, called caspases. There are two different pathways: intrinsic and extrinsic. Apoptosis via both the extrinsic Fas-ligand/TNF receptor pathway or the intrinsic cytochrome c release pathway leads to activation of caspase-3. Therefore immunocytochemistry with antibody to activated caspase-3 (Santa Cruz Biotechnology, Santa Cruz, Calif.) was performed on REH cells, 4 weeks after transfection. The results are shown in FIG. 4, which shows Caspase-3 expression 4 weeks after transfection, caspase-8 expression 4 weeks after transfection, and FLASH expression 3 weeks after transfection. REH cells with pLNCX2 plasmid showed no staining with antibody to activated caspase-3 (A, top left), activated caspase-8 (C, middle left), or FLASH (E, bottom left). In contrast, the REH cells with pLNCX2-HOXB4 plasmid show deep staining with the anti-caspase-3 (B, top right), anti-caspase-8 (D, middle right), and anti-FLASH antibodies (F, bottom right). Negative and positive control antibody stains were similar to FIG. 2. REH cells transfected with pLNCX2-HOXB4-IRES-GFP expressed activated caspase-3, compared to pLNCX2-IRES-GFP-transfected controls that showed no activated caspase-3 expression. At 3 weeks there was faint staining with anti-caspase-3 antibody. At 2 weeks there was none.

Next, we performed “real-time” RT-PCR to examine whether FLASH RNA was upregulated. HOXB4 transfectants demonstrated elevation of FLASH RNA compared to controls at different time points after transfection. Therefore, we performed immunocytochemistry with FLASH antibody (Imgenex, San Diego, Calif.) to see if our HOXB4-transfected cells also exhibited increased FLASH expression compared to controls. FLASH expression could be detected faintly at 2 weeks and positively at 3 and 4 weeks (FIG. 4).

To confirm this pathway, we tested for the presence of the intermediary caspases-8. HOXB4-transfected REH cells express activated caspases-8 in a similar time sequence. At 4 weeks, there was activated caspases-8 expression (FIG. 4). At 3 weeks, there was faint expression, and at 2 weeks there was no detectable activated caspases-8 (data not shown).

Our results also showed that REH cells overexpressing HOXB4 do not express increased caspase-9 and SMAC. To investigate if the intrinsic pathway was also active, we performed immunocytochemistry with an anti-caspase-9 (activated) antibody at 4 weeks and anti-SMAC antibody at 3 weeks. There was no difference between REH cells overexpressing HOXB4 and control transfectants (FIG. 5). FIG. 5 shows Caspase-9 expression 4 weeks after transfection and SMAC expression 3 weeks after transfection. REH cells with pLNCX2 plasmid (left top and bottom, A and C) showed no staining with antibodies to activated caspase-9 or SMAC. REH cells with pLNCX2-HOXB4 plasmid also showed no staining with anti-caspase-9 or anti-SMAC antibodies. Negative and positive control antibody stains were similar to FIG. 2. There was also no difference at all other time points studied (2, 3, and 4 weeks, data not shown).

Our results also showed that these effects are generalizability to other malignant cell types. To determine if the apoptotic effect of HOXB4 was specific to the REH cells only, or was more widespread, we transfected WSU-DLCL2 cells with HOXB4.

The REH cell represents a very early B cell precursor. Further stages of B cell maturation culminate in the plasma cell. Malignancy can appear at any stage of the differentiation pathway, leading to various clinical phenotypes. Diffuse large cell lymphoma represents a malignancy derived from a more mature B cell. We have successfully created a cell line from diffuse large cell lymphoma at Wayne State University, characterized it, and published it as WSU-DLCL2.

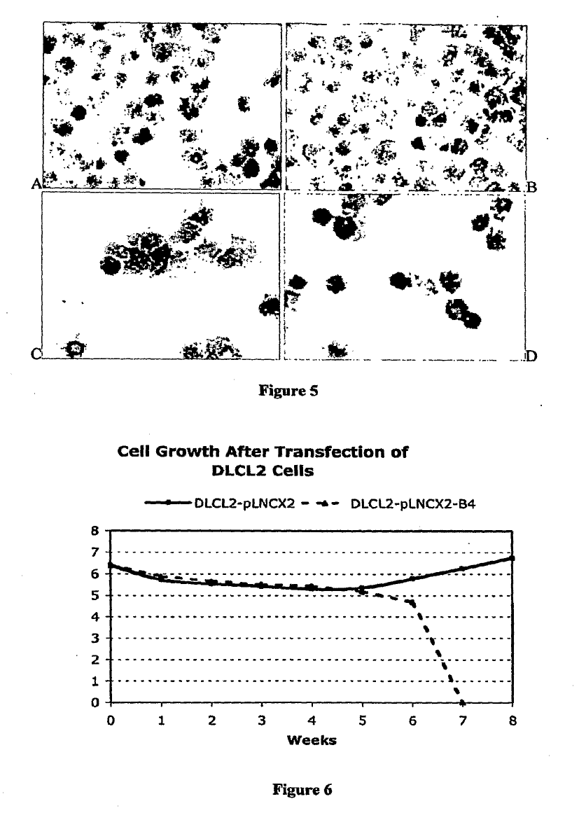

Data from similar HOXB4 transfection experiments demonstrate similar effects in this cell line. Two and a half million DLCL2 cells were transfected with pLNCX2-HOXB4 and control plasmid. A cell growth curve is shown in FIG. 6 that confirms cell death as a consequence of HOXB4 overexpression. FIG. 6 shows cell death in WSU-DLCL2 cells with HOXB4 over-expression. Two and a half million cells from the WSU-DLCL2 cell line were transfected with pLNCX2-HOXB4 and control plasmid. The HOXB4 transfectants died after 6 weeks while the control transfectants survived. WSU-DLCL2 has been examined for DNA disintegration by TUNEL assay and has shown results similar to REH cells (data not shown).

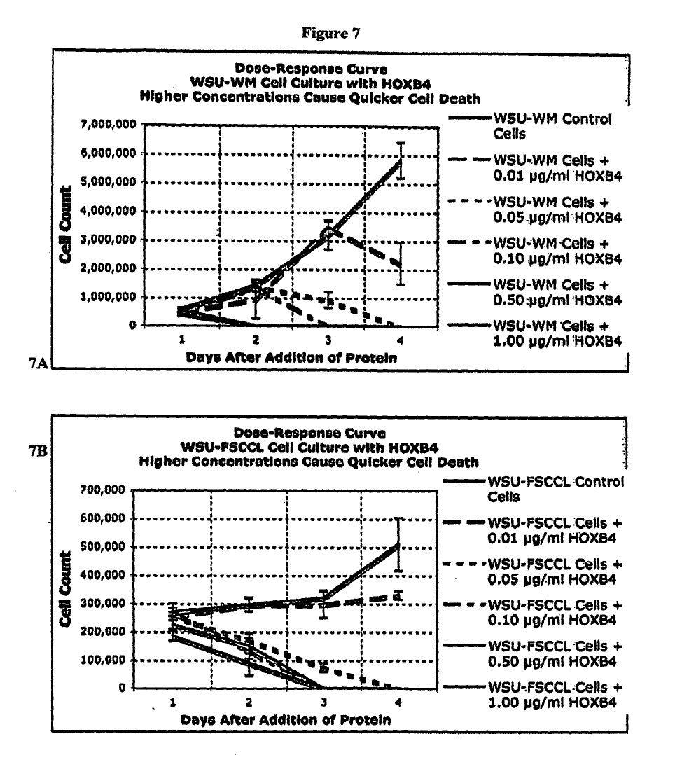

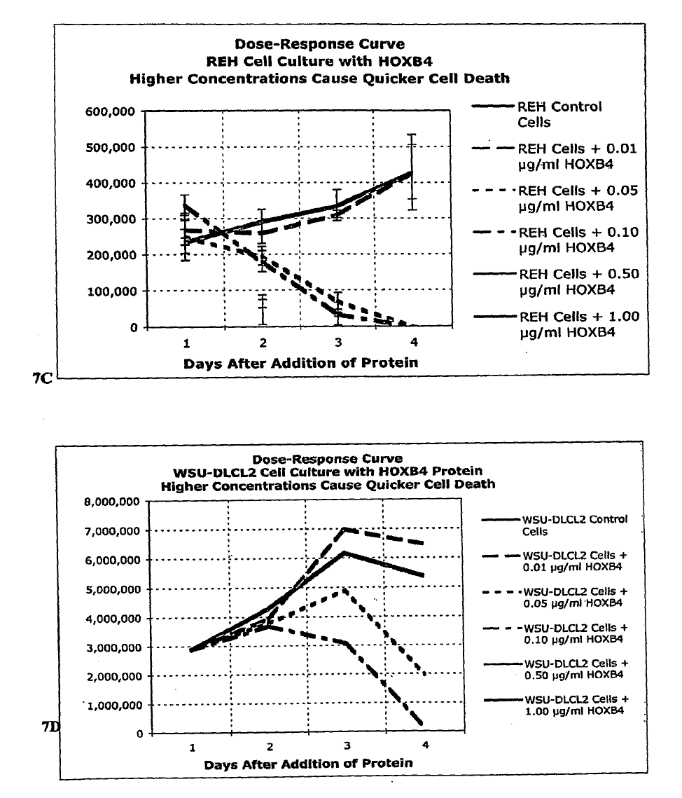

Our tests demonstrate that HOXB4 causes apoptosis in REH cells, and provide evidence for apoptosis in one other malignant B cell line. We had expected the opposite effect, but at least in our experimental model, the apoptotic effect of HOXB4 predominates over the proliferative effect. We determined that higher concentration of HOXB4 resulted in quicker cell deaths among all cell lines (FIGS. 7(A)-(D)).

An atypical feature of our model of apoptosis is the 3- to 4-week delay in the onset of apoptosis. Both REH as well as WSU-DLCL2 cells demonstrate no substantial difference in cell counts between HOXB4 transfectants and control for several weeks. HOXB4 expression is demonstrable at 2 weeks, but the cascade of apoptosis only begins once the cells enter exponential growth. Therefore it would appear that the onset of HOXB4-induced apoptosis is linked to the entry of REH and WSU-DLCL2 cells into exponential growth after recovery from the effects of transfection.

It was recently demonstrated that if HOXB4 protein is added to the culture medium of HSCs, it enters the cells and causes clonal expansion of HSCs. This is because cultured cells take up HOXB4 via its 16-amino acid third helix of the DNA-binding homeodomain by passive translocation in a receptor-independent fashion.

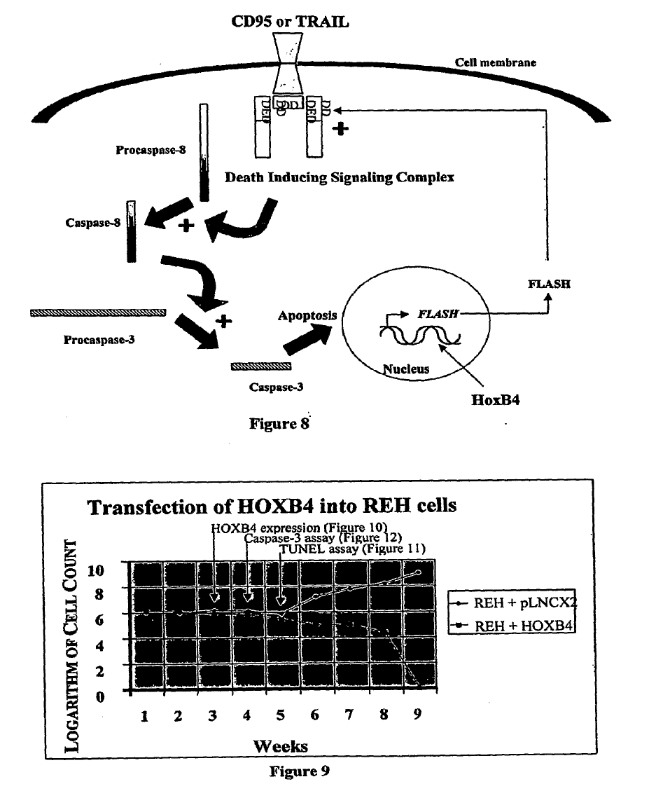

We currently believe that a mechanism of apoptosis induced by overexpression of HOXB4 in REH cells is similar to that in the notochord of Xenopus. The hoxb4 RNA is translated into HOXB4 protein that enters the nucleus and causes the transcription of the FLASH gene, which is subsequently translated into FLASH protein. The FLASH protein then interacts with the death-inducing signaling complex and activates caspase-8. The usual cascade of apoptosis begins, culminating in cell death (FIG. 8). FIG. 8 shows a possible mechanism of HoxB4-induced apoptosis in REH cells. The HOXB4 RNA transcribed from pLNCX2-HOXB4-IRES-GFP is translated into HOXB4 protein. This protein enters the nucleus and turns on the transcription of FLASH RNA that is translated into FLASH protein. This protein is a part of the death-inducing signaling complex. It cleaves procaspase-8 to caspase-8, leading to apoptosis.

The temporal order of HOXB4, FLASH, activated caspase-8, and activated caspase-3 expression with DNA cleavage (Table 1) is consistent with this mechanism.

TABLE 1

| Assay | 2 weeks | 3 weeks | 4 weeks |
|---|---|---|---|
| HoxB4 | + | + | + |
| FLASH | ± | + | + |
| Caspase-8 | − | ± | + |
| Caspase-3 | − | ± | + |
| TUNEL | − | ± | + |
| Caspase-9 | − | − | − |
| SMAC | − | − | − |

Table 1 demonstrates a time course of events culminating in DNA disintegration. HOXB4 was the first protein to appear. This could be detected as early as 2 weeks after transfection and remained positive as long as 4 weeks after transfection. Caspase-8-associated protein 2 (FLASH) was faintly detectable at 2 weeks but became positive at 3 weeks and remained positive at 4 weeks. Activated caspase-3, activated caspase-8 and TUNEL were not detectable at 2 weeks, faintly detectable at 3 weeks, and positively at 4 weeks. This time course is consistent with the proposed mechanism (FIG. 8). Caspase-9 and SMAC were negative at 2, 3, and 4 weeks.

However, alternative mechanisms could also have been possible. As one example only, Deformed, the Drosophila homolog of HOXB4, activates Reaper. The human homolog of Reaper is the second mitochondria-derived activator of caspase (SMAC, also called DIABLO—direct IAP-binding protein with low pI). SMAC binds to the BIR3 domain of XIAP (X-linked inhibitor of apoptosis), and thereby relieves the inhibition of caspase-9 activity caused by XIAP. SMAC, like cytochrome c, is released from mitochondria during apoptosis. Since both SMAC and caspase-9 are not detectable in REH cells that overexpress HOXB4, we do not believe that this pathway is involved in the observed apoptosis. To our knowledge, this is the first observation of HOXB4 causing apoptosis in human cells.

We performed tests using a HOXB4 gene in a retroviral vector spliced to an internal ribosome entry site (IRES) and green fluorescent protein (GFP), as used in a recent paper showing retrovirally-mediated clonal expansion of HSCs (Antonchuk et al., 2002). The plasmid vector was digested to release the HOXB4-IRES-GFP expression cassette and ligated it into the pLNCX2 expression vector (Stratagene, La Jolla, Calif.). The ligation of the insert was confirmed by restriction digest and also by sequence analysis. Using lipofection, the REH cell line was transfected with the pLNCX2-HOXB4-IRES-GFP plasmid, with the native pLNCX2 as control.

Both electroporation and lipofection were used to transfect the pLNCX2-HOXB4-IRES-GFP vector into REH cells. Lipofection produced better results and was optimized. After a typical lipofection, it took approximately 3-4 weeks for REH cells to recover and begin exponential growth. As expected, the REH cells transfected with pLNCX2 and began to grow and entered exponential phase after 4 weeks. The REH cells transfected with pLNCX2-HOXB4-IRES-GFP followed the same quiescent path as the pLNCX2 transfected cells for 4 weeks, but instead of entering an exponential phase, inexplicably died.

Repeating the experiment several times produced the same result. Deleting the IRES-GFP segment (pLNCX2-HOXB4) had no effect: the cells died again. Deleting the HOXB4 gene (pLNCX2-IRES-GFP) abrogated the effect and the cells behaved just like the controls, and survived. After a total of 5 attempts that only confirmed cell death, this effect was investigated further.

Cell Growth Curve: To quantify the phenomenon of cell death, the transfected cells were repeatedly sampled at weekly intervals and counted. FIG. 9 shows a typical cell growth curve of REH cells transfected with pLNCX2 control plasmid and pLNCX2-HOXB4-IRES-GFP construct. The cell counts remained close to each other for about 4 weeks and then began to diverge. As the control cells entered the exponential phase, the HOXB4 cells entered the apoptotic phase. Results with pLNCX2-HOXB4 plasmid without the IRES-GFP were similar to the pLNCX2-HOXB4-IRES-GFP plasmid and results of pLNCX2-IRES-GFP were similar to pLNCX2 (data not shown). The growth curves for HOXB4 transfectants and control remain superimposable until cells recovered from the effects of transfection. Then the cells tried to enter exponential growth. At this point, the HOXB4 transfectants started to die and were dead in a few weeks. The control cells grew exponentially. Repeated experiments showed similar results.

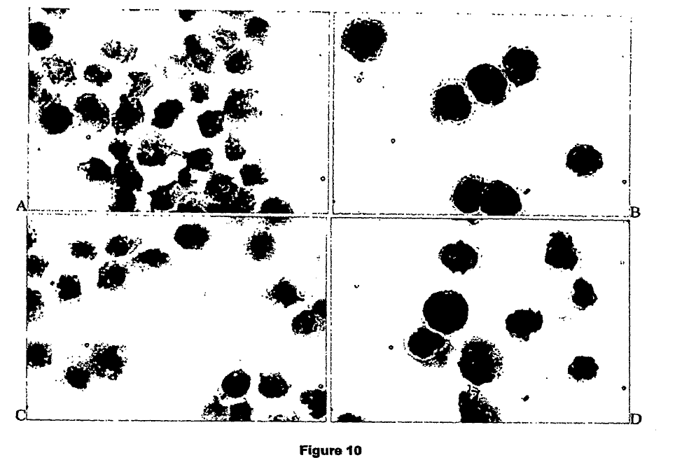

Protein Expression: To determine if this effect of cell death was attributable to HOXB4 protein, the cells transfected with pLNCX2-HOXB4-IRES-GFP were examined under a fluorescent microscope. The transfected cells were fluorescent, indicating that GFP was being expressed via the IRES (data not shown). A HOXB4 rat monoclonal antibody was obtained from the Developmental Studies Hybridoma Bank at the University of Iowa and immunocytochemistry was performed on Cytospin specimens of REH cells 2 to 4 weeks after transfection (FIG. 10). FIG. 10 shows immunocytochemistry with HOXB4 antibody 2 weeks after transfection. The top left panel A shows REH cells transfected with control plasmid pLNCX2 and stained with anti-HOXB4 antibody. The top right panel B shows REH cells transfected with pLNCX2-HOXB4-IRES-GFP plasmid and stained similarly. The bottom left panel C shows REH cells transfected with pLNCX2-HOXB4-IRES-GFP plasmid and stained with rat IgG (negative control). The bottom right panel D shows REH cells transfected with pLNCX2-HOXB4-IRES-GFP plasmid stained with anti-CD45RO (leukocyte common antigen) antibody (positive control). REH cells transfected with native pLNCX2 plasmid showed similar results with both control antibodies (micrographs not shown).

As expected, the REH cells stained with the anti-HOXB4 monoclonal antibody 2 weeks after transfection. Similar results were obtained at 3 and 4 weeks (data not shown). The cells transfected with pLNCX2 had no HOXB4 staining. Our hypothesis that HOXB4 would cause proliferation of REH cells had been disproved. Given the established role of homeobox proteins in apoptosis, we then hypothesized that HOXB4 was causing apoptosis in REH cells.

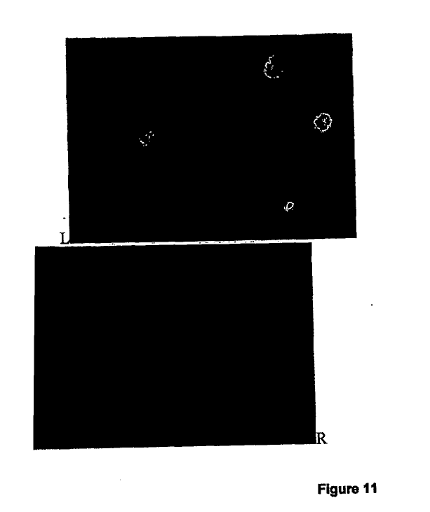

TUNEL Assay: To investigate whether the observed cell death was due to apoptosis, a TdT-mediated dUTP nick-end labeling (TUNEL) assay was performed to examine DNA fragmentation. The IRES-GFP portion of the plasmid was deleted for this series of experiments to prevent interference with fluorescence detection. The results are shown in FIG. 11, which shows a TUNEL assay after 4 weeks of transfection: REH cells with native pLNCX2 plasmid (left) demonstrated no DNA fragmentation with red fluorescence. In contrast, the REH cells with pLNCX2-HOXB4-IRES-GFP plasmid (right) demonstrated cell death (green fluorescence) and DNA fragmentation (yellow fluorescence). After 4 weeks, the REH cells transfected with pLNCX2-HOXB4 had DNA fragmentation, a feature of apoptosis, in contrast to control cells that had no DNA fragmentation. DNA fragmentation could be faintly demonstrated in pLNCX2-HOXB4 transfected cells at 3 weeks, but not before (data not shown).

Caspase-3 Assay: To confirm that enzymes that mediate apoptosis were present, a caspase-3 assay was performed. Apoptosis is a carefully orchestrated, active, metabolic process that is mediated via aspartate-specific cysteine proteases, caspases. There are two different pathways: intrinsic and extrinsic. Apoptosis via both the extrinsic Fas-ligand/TNF receptor pathway or the intrinsic cytochrome c release pathway leads to activation of caspase-3.

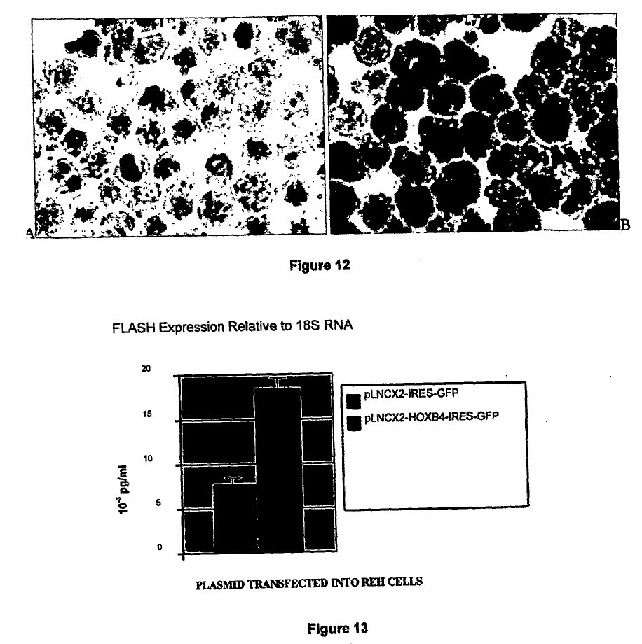

Therefore immunocytochemistry was performed on REH cells with antibody to activated caspase-3 (Santa Cruz Biotechnology, Santa Cruz, Calif.), 4 weeks after transfection. The results are shown in FIG. 12, which shows Caspase-3 expression after 4 weeks of transfection. REH cells with pLNCX2 plasmid (A, left) demonstrate no staining with antibody to activated caspase-3. In contrast, the REH cells with pLNCX2-HOXB4 plasmid (B, right) show deep staining with the same antibody. Negative and positive control antibody stains are similar to FIG. 10. REH cells transfected with pLNCX2-HOXB4-IRES-GFP expressed caspase-3, compared to pLNCX2-transfected controls that showed no caspase-3 expression. At 3 weeks, there was faint caspase-3 expression and at 2 weeks there was none (data not shown).

FLASH Assay: Apoptosis caused by HOXB4 is mediated via direct regulation of FLASH (FLICE-associated huge protein), also known as caspase-8 associated protein 2 (Casp8ap2). We therefore performed “real-time” RT-PCR for FLASH in HOXB4 transfected REH cells and controls at 4 weeks. There was threefold overexpression of FLASH (FIG. 13). FIG. 13 shows FLASH RNA expression in HOXB4 transfected REH cells and controls. FLASH expression is upregulated by a factor of 3 in HOXB4 transfectants.

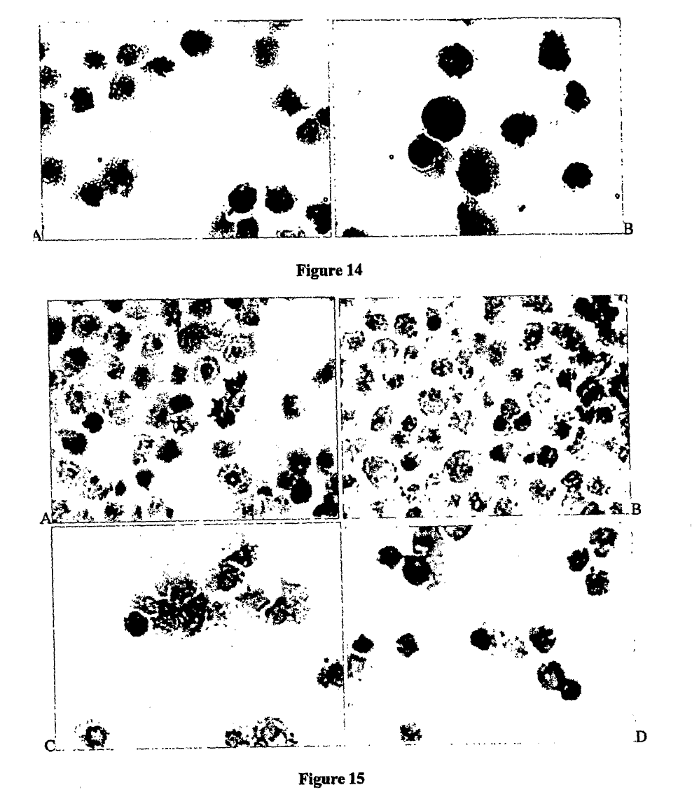

Next, we performed immunocytochemistry for FLASH on REH transfectants using a FLASH antibody (Imgenex, San Diego, Calif.). HOXB4 transfectants demonstrated FLASH expression at 3 weeks (FIG. 14). FIG. 14 shows FLASH expression after 3 weeks of transfection. REH cells with pLNCX2 plasmid (left) demonstrate no staining with antibody to FLASH. In contrast, the REH cells with pLNCX2-HOXB4 plasmid (right) show deep staining with the same antibody. Negative and positive control antibody stains were similar to FIG. 10. FLASH expression could also be detected at 4 weeks, but only faintly at 2 weeks (data not shown).

These findings are summarized in Table 2.

TABLE 2
Time course of events culminating in DNA disintegration.

| Assay | 2 weeks | 3 weeks | 4 weeks |
|---|---|---|---|
| HOXB4 | + | + | + |
| FLASH | ± | + | + |
| Caspase-3 | − | ± | + |
| TUNEL | − | ± | + |

Alternate Mechanism of Apoptosis: In Drosophila, the gene homologous to HOXB4 is called Deformed. The Deformed gene causes apoptosis via activation of a gene called Reaper. A close human homolog of Reaper is second mitochondria-derived activator of caspase (SMAC, also called DIABLO, direct IAP-binding protein with low pI). SMAC shares a conserved 4-amino acid sequence—alanine-valine-proline-isoleucine—with Reaper that binds to the BIR3 domain of XIAP (X-linked inhibitor of apoptosis), and thereby relieves the inhibition of caspase-9 activity caused by XIAP. SMAC is released from mitochondria during apoptosis in a manner similar to cytochrome c. It was possible that HOXB4 was causing apoptosis via SMAC, either exclusively or in a process that is parallel to the FLASH pathway.

SMAC and Caspase-3 Assays: To investigate if the alternative mechanism was also active, we performed immunocytochemistry with an anti-caspase-9 (activated) antibody at 4 weeks and anti-SMAC antibody at 3 weeks. There was no difference between REH cells overexpressing HOXB4 and control transfectants (FIG. 15). FIG. 15 shows Caspase-9 expression 4 weeks after transfection and SMAC expression 3 weeks after transfection. REH cells with pLNCX2 plasmid (left top and bottom, A and C) showed no staining with antibodies to activated caspase-9 or SMAC. REH cells with pLNCX2-HOXB4 plasmid also showed no staining with anti-caspase-9 or anti-SMAC antibodies. There was also no difference at all other time points studied (2, 3, and 4 weeks, data not shown).

Our unexpected findings may appear contradictory and conflicting, but are not without precedent in the homeobox literature. Presumably there are important differences between hematopoietic stem cells and REH cells that are recognized by HOXB4. It has been hypothesized that the divergent effects of HOXB4 may occur because HOXB4-induced transcription may require additional factors to bind to adjacent gene enhancer sites, and that these factors may only be available in specific cells and tissues.

Examining the classical description of the divergent homeobox gene, Aniridia, throws more light on the subject. Effects of homeobox genes are dependent on cell type and their stage of differentiation. The Aniridia effect is sensitive to the developmental stage of the embryo, and it is either observed, or not observed. Our effect is sensitive to cell type and is observed in pre-B-ALL REH cells, or its converse occurs in a different cell type, the HSCs.

Homeobox proteins are able to passively translocate across cell membranes in a receptor-independent fashion. This has been demonstrated in the case of HOXB4 by adding it to the culture medium of HSCs. HSCs proliferate in the same manner as when there is retrovirally mediated overexpression. The HOXB4 protein can also be fused with the HIV TAT protein, and this fusion construct can also passively translocate across the cell membrane. This is because cells in culture take up the protein via the 16-amino acid third helix of the DNA-binding homeodomain of homeobox proteins by passive translocation in a receptor-independent fashion. Presumably, gradients of homeobox protein concentration exist in tissues that affect their development. Therefore, we hypothesized that addition of HOXB4 protein into cultures of hematological malignancies will result in apoptosis. Furthermore, injection of HOXB4 protein into animals should show similar results.

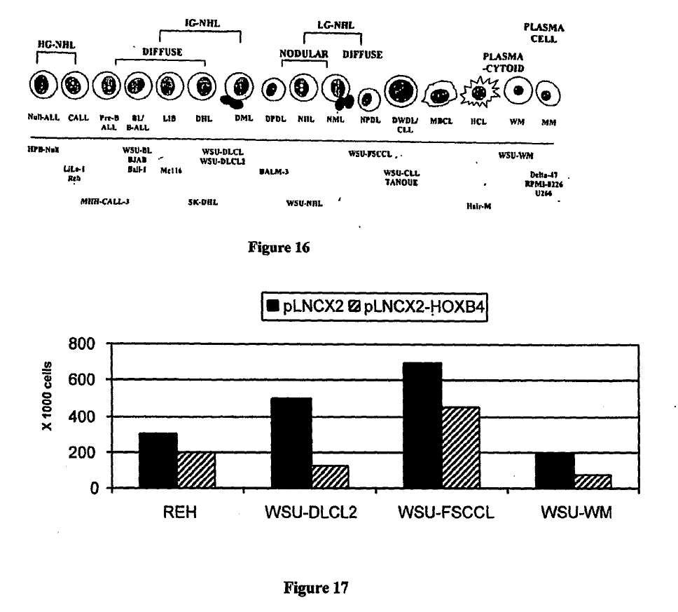

To determine if the apoptotic effect of HOXB4 was specific to the REH cells only, or was more widespread, we transfected other cell types with HOXB4. The REH cell represents a very early B cell precursor. Further stages of B cell maturation culminate in the plasma cell. Malignancy can appear at any stage of the differentiation pathway, leading to various clinical phenotypes. Diffuse large cell lymphoma, follicular small cleaved cell lymphoma, and Waldenström macroglobulinemia represent successively mature B cell malignancies. We have successfully created cell lines from these phenotypes and characterized them as WSU-DLCL2, WSU-FSCCL, and WSU-WM, respectively. Along with the REH cell line, these lines represent almost the entire spectrum of B cell malignancies (e.g., FIG. 16).

As the B cell matures, it becomes more difficult to successfully transfect cell lines derived from it. We transfected the WSU-DLCL2, WSU-FSCCL, and WSU-WM cell lines with HOXB4 and demonstrated similar effects along the entire B cell spectrum. Two and a half million cells of each cell line were transfected with pLNCX2-HOXB4 and control plasmid. At four weeks, all cell lines demonstrate reduced growth with HOXB4 overexpression, as shown in FIG. 17, where all cell lines demonstrate reduced growth at 4 weeks with over-expression of HOXB4, indicating that the presumed apoptosis is a general effect across a spectrum of B cell malignancies. The WSU-DLCL2 cell line has been examined for cell growth and DNA disintegration. This cell line demonstrates results similar to REH cells (data not shown).

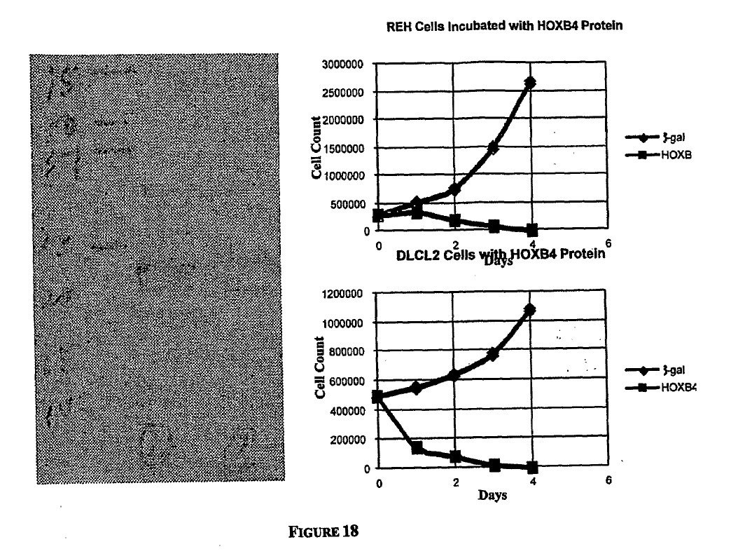

We also synthesized HOXB4 protein by bacterial expression. The HOXB4 gene was ligated into the pBAD/HIS C plasmid (Invitrogen, Carlsbad, Calif.) in frame with the 6X histidine leader sequence. TOP10 E. coli bacteria (Invitrogen, Carlsbad, Calif.) were transformed with the pBAD/HIS-HOXB4 plasmid and the HOXB4 protein was induced with L-arabinose. After 2 hours, the histidine-tagged HOXB4 protein was purified by a nickel column, Ni-NTA Magnetic Agarose Beads (QIAGEN, Valencia, Calif.), using native conditions. Confirmation of protein production was obtained by Western blot using the anti-HOXB4 antibody. As predicted for HOXB4, an approximately 25 kD band was readily detectable in successive column eluates. FIG. 18, shows production of HOXB4 protein and addition of protein to culture of REH and DLCL2 cells. The left panel shows a band visible just below the 25-kD marker. This is the predicted weight of HOXB4. The top right panel shows addition of HOXB4 protein and control β-galactosidase protein to REH cells at a concentration of 0.2 μg/ml. After a slight growth at day 1, the cells die rapidly with daily addition of HOXB4 protein. The bottom right panel shows DLCL2 cells given 2 μg/ml every day undergo rapid cell death. Similarly, the LacZ gene was also ligated into the same plasmid, and used to produce β-galactosidase as a control protein (data not shown).

The HOXB4 protein was added daily to the culture of REH and DLCL2 cells at varying concentrations. Both of these cell lines undergo cell death in a matter of days (FIG. 18), rather than weeks with the transfection model. We expect that the HOXB4 protein is entering the REH and DLCL2 cell lines via passive translocation and causing the FLASH-mediated activation of caspase-8 leading to DNA disintegration.

### EXAMPLE 2

We have demonstrated that the REH cell line, derived from acute lymphoblastic leukemia, when transfected with the HOXB4 gene undergoes apoptosis, or programmed cell death. That is to say, when this cell line overexpresses the HOXB4 gene, it kills itself. We have further demonstrated that this cellular suicide happens because a gene called FLASH is activated, which in turn activates an enzyme called caspase-8. This enzyme then activates caspase-3, which then leads to the usual cascade of apoptosis leading to destruction of the cell's chromosomes and disintegration of the DNA.

The HOXB4 protein is known to go through cell membranes without any difficulty. We therefore synthesized the HOXB4 protein in bacteria, and purified it. We then demonstrated that when HOXB4 protein is added to the culture medium of REH cells at a concentration of 0.2 μg/ml, these cells die in a similar manner.

We also created an animal model of treatment of human leukemia and evaluated the effect of HOXB4 overexpression. REH cells form tumors in SCID

(immunocompromised) mice. We injected 15 million REH cells each into both flanks of 3 SCID mice. Tumors typically develop in 3 to 4 weeks. After one week, we injected the right flank of each mouse with 2 μg of HOXB4 protein. The left flank served as control. The mice have been followed for 4 weeks. None of the mice have developed any tumors, either on the right flank, where the protein was injected, or on the left flank. This indicates that not only HOXB4 was able to kill the REH cells in the right flank, it was also able to circulate in the body, reach the left side, and kill the cancer cells on the left side. Therefore, no tumors have been observed.

We also discovered unexpectedly that when WSU-DLCL2 cells (diffuse large cell lymphoma) are exposed to HOXB4, they also undergo apoptosis. Similarly, WSU-WM cells (Wäldenstrom's macroglobulinemia) and WSU-FSCCL (follicular small cleaved cell lymphoma) cells also demonstrate cell death. This means that HOXB4 is active against many forms of hematological malignancies.

We have also shown that when kidney embryonal carcinoma cells overproduce HOXB4 protein, they also kill themselves. Therefore the effect of HOXB4 is not restricted to hematological malignancies alone.

A tumor transplantation model was created for propagation of DLCL2 tumors derived from the DLCL2 cell line. Using a 12 G trocar needle, 20 to 30 mg of DLCL2 tumor was introduced into bilateral flanks of 6 SCID mice. All 6 mice developed tumors in both flanks after 10 days. Then 2 μg of HOXB4 protein was injected every day into the right flank of 3 mice. Approximately 25 μl was injected into the tumor itself, and the rest (approximately 225 μl) was injected into the surrounding subcutaneous tissue. The other 3 mice were injected with 2 μg of β-galactosidase into the right flank tumors in a similar manner. The β-galactosidase protein had been produced from the LacZ gene in exactly the same maimer as HOXB4 protein production. The injections were continued daily for 5 days. Since the act of injecting itself increases the size of the tumors, we allowed the tumors to grow for another one week before measuring tumor size.

The tumors were measured in length and breadth, but thickness was difficult to measure. Therefore tumor size is expressed in mm² below. Prior to injection, the tumor sizes were not significantly different (Table 3). After injection, by visual inspection, the tumors on the right flank of the HOXB4-injected mice were much smaller than those on the left. In contrast, no difference in tumor size was observed in the β-galactosidase group.

TABLE 3
Tumor sizes before injection.

| | HOXB4 | No injection | | | β-galactosidase | No injection | |
|---|---|---|---|---|---|---|---|
| | Right flank | Left flank | p value | | Right flank | Left flank | p value |
| Mouse 1 | 12 | 30 | | Mouse 4 | 48 | 30 | |
| Mouse 2 | 24 | 0 | | Mouse 5 | 25 | 16 | |
| Mouse 3 | 16 | 36 | | Mouse 6 | 24 | 0 | |
| Mean | 17.3 | 22 | 0.7099 | Mean | 32 | 15.3 | 0.1097 |

The sizes were compared using a two-tailed, unpaired, homoscedastic t test. The results are listed in the table below (Table 4). The tumors on the right flank in the HOXB4-treated group were about half the side of the left flank. This difference was significant with a p value of 0.0492. The corresponding p value for the β-galactosidase group was 0.4481, indicating no significant difference.

TABLE 4
Tumor sizes after injection.

| | HOXB4 | No injection | | | β-galactosidase | No injection | |
|---|---|---|---|---|---|---|---|
| | Right flank | Left flank | p value | | Right flank | Left flank | p value |
| Mouse 1 | 25 | 126 | | Mouse 4 | 90 | 90 | |
| Mouse 2 | 72 | 99 | | Mouse 5 | 42 | 42 | |
| Mouse 3 | 80 | 110 | | Mouse 6 | 100 | 90 | |
| Mean | 59 | 112 | 0.0492 | Mean | 77 | 74 | 0.4481 |

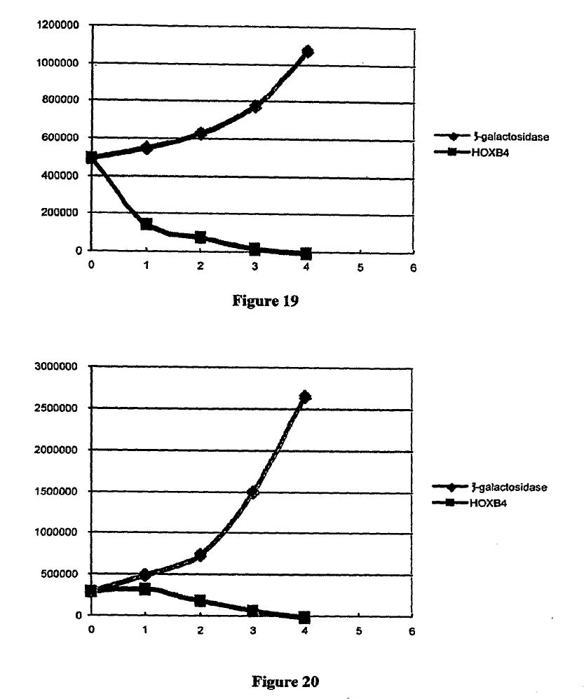

The DLCL2 cell line was routinely cultured in RPMI medium with β-galactosidase and HOXB4 protein at concentrations of 2 μg/ml. Cells were counted each day. Cell growth curves are depicted below (FIG. 19). The HOXB4-treated all died within 4 days, while the β-galactosidase-treated group continued their growth in the usual exponential manner.

The REH cell line was also cultured in vitro in a similar manner and exposed to HOXB4 and β-galactosidase at concentrations of 0.2 μg/ml. The results are shown below. The HOXB4-treated cells all died in 4 days while the β-galactosidase-treated group continued their growth in the usual exponential manner (FIG. 20).

The cancer cell line 293 is derived from kidney embryonal cells. A modification of this cell line containing ecdysone receptors is available from Stratagene (La Jolla, Calif.). This cell line, ER-293, was transfected with the pEGSH plasmid containing the HOXB4 cDNA gene. Several cell lines were isolated from the transfectants. Two of these, ER-293-HOXB4 #6 and ER-293-HOXB4#8 were then exposed to ponasterone A. Ponasterone A is an ecdysone analog that causes the expression of any gene contained in the pEGSH plasmid. In this model, ponasterone causes overexpression of HOXB4.

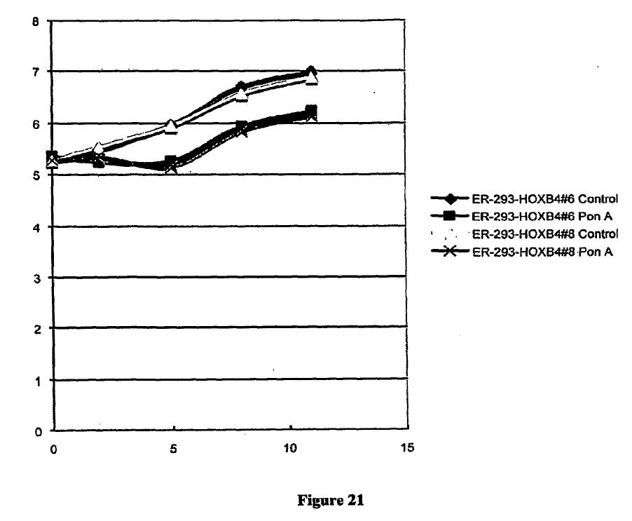

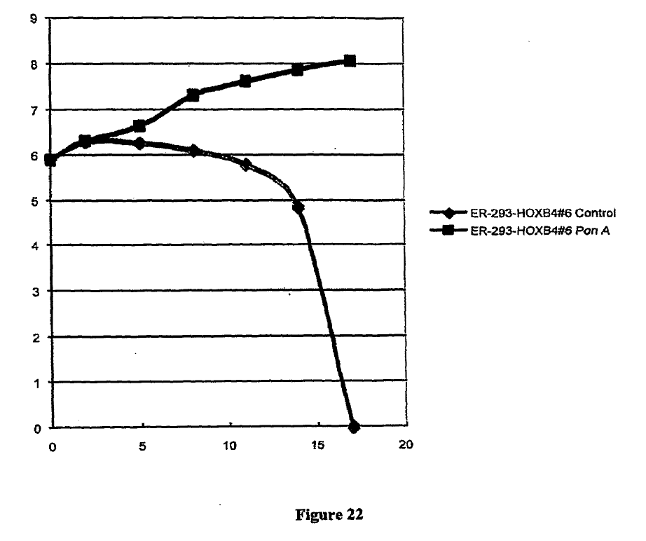

As shown in FIG. 21, the cell lines demonstrated growth inhibition, but then escaped the effect of HOXB4 and began exponential growth. Immunocytochemistry demonstrated that HOXB4 was expressed at day 3, but expression declined by day 10. We therefore hypothesized that the HOXB4 gene had been silenced by methylation. This if the cells were cultured with demythylating agents before addition of ponasterone A, the ER-293 cells should undergo complete apoptosis. Therefore the ER-293-HOXB4#6 cell line was cultured with 5-azacytidine (1 μM) and trichostatin A (0.2 μM). The cell line continued to grow normally. On day 2, ponasterone A was added to the cell line and its growth followed. As shown in FIG. 22, compared to control cell line, where no ponasterone A was added, the ER-293-HOXB4#6 cell line died completely. We currently believe that HOXB4 expression is suppressed by the ER-293 cell lines to preserve its own survival. This suppression is abrogated by demethylating agents. We predict that addition of HOXB4 protein to the culture medium of ER-293 cells will cause it to undergo apoptosis, just like REH and DLCL2 cells. Tumors derived from kidney embryonal tissue may also be amenable to treatment with HOXB4.

REH, WSU-DLCL2, WSU-FSCCL, and WSU-WM cells form tumors when injected subcutaneously in SCID mice. Therefore we obtained SCID mice and injected bilateral flanks with 20 to 30 mg of WSU-DLCL2 tumor transplanted from other SCID mice used for propagation. Tumors grew in both flanks and were measured. HOXB4 protein (2 μg) was injected into and around the tumor in the right flank of 3 SCID mice. Similarly, β-galactosidase (2 μg) was injected into the right flank of 3 control mice.

Each animal received an injection every day for 5 days. Since the act of injection itself can increase the measured size of the tumor, we waited one week before measuring the final tumor size. The tumor size measured in mm was converted to mg using our previous experience. The results are depicted in Table 5.

TABLE 5

| | | Tumor | | Tumor |
|---|---|---|---|---|
| | Tumor Size | Weight | Tumor Size | Weight |
| | Right (mm) | Right (mg) | Left (mm) | Left (mg) |
| Mice Before | | | | |
| HOXB4 Injection | | | | |
| Mouse 1 | 4 × 3 | 18 | 5 × 6 | 75 |
| Mouse 2 | 6 × 4 | 48 | 0 × 0 | 0 |
| Mouse 3 | 4 × 4 | 32 | 6 × 6 | 108 |
| Mean | | 17.3 | | 61 |
| t test p value | 0.440 | | | |
| Mice After | | | | |
| HOXB4 Injection | | | | |
| Mouse 1 | 5 × 5 | 63 | 14 × 9 | 557 |
| Mouse 2 | 8 × 9 | 288 | 9 × 11 | 446 |
| Mouse 3 | 10 × 8 | 320 | 10 × 11 | 550 |
| Mean | | 223.7 | | 517.7 |
| t test p value | 0.029 | | | |
| Mice Before | | | | |
| β-gal Injection | | | | |
| Mouse 4 | 8 × 6 | 144 | 5 × 6 | 75 |
| Mouse 5 | 5 × 5 | 63 | 4 × 4 | 32 |
| Mouse 6 | 4 × 6 | 48 | 0 × 0 | 0 |
| Mean | | 85 | | 35.7 |
| t test p value | 0.126 | | | |
| Mice After | | | | |
| β-gal Injection | | | | |
| Mouse 4 | 9 × 10 | 405 | 10 × 9 | 405 |
| Mouse 5 | 6 × 7 | 126 | 7 × 7 | 172 |
| Mouse 6 | 10 × 10 | 500 | 9 × 10 | 405 |
| Mean | | 344 | | 327.3 |

| | |
|---|---|
| t test p value | 0.455 |

Table 5 shows bilateral tumor sizes in HOXB4 and β-galactosidase injected mice before and after injection. Each mouse was injected with 2 μg of each protein in the right flank only. The β-galactosidase group demonstrates no difference in tumor sizes either before or after injection. The HOXB4 group demonstrates no difference in tumor sizes before injection, but the tumor size after injection in the right flank is smaller than the left. There was no difference in tumor size between the right and left side in the HOXB4 injected mice before the injections. After the injections, the tumor size was visibly smaller. Even with only 3 mice and using a low dose of 2 μg (we are currently testing 20 μg) a two-tailed, unpaired t test demonstrates a statistically significant difference. No difference was noted for the β-galactosidase group. (Tumor sizes measured in square mm also demonstrate similar significance.)

We detected no apparent ill effects in the tested animals from the protein injections. Their behavior has continued to be normal, and they have continued to feed normally.

We hypothesize that a mechanism of apoptosis induced by overexpression of HOXB4 in REH cells is similar to that in the notochord of Xenopus. The HOXB4 RNA is translated into HOXB4 protein that enters the nucleus and causes the transcription of the FLASH gene, which is subsequently translated into FLASH protein. The FLASH protein then interacts with the death-inducing signaling complex and activates caspase-8. The usual cascade of apoptosis begins, culminating in cell death (FIG. 8). FIG. 8 shows a proposed mechanism of HOXB4-induced apoptosis in REH cells. The HOXB4 RNA transcribed from pLNCX2-HOXB4-IRES-GFP is translated into HOXB4 protein that enters the nucleus and turns on the transcription of FLASH RNA that is translated into FLASH protein (bottom right). This protein is a part of the death-inducing signaling complex (DISC) (top left). This cleaves procaspase-8 to caspase-8, leading to apoptosis. (Alternative mechanisms are also possible).

Our finding that HOXB4 causes apoptosis also answers a question that has not been asked so far. We know very well that HOXB4 when transfected into HSCs causes clonal expansion, and that when these cells are transplanted, the HSC compartment of a mouse is reconstituted. Both lymphoid and myeloid lines are generated, but the HSCs do not grow beyond their normal limits and continue to respect their regulatory limits. This is in sharp contrast to other homeobox genes such as HOXB8 that, in conjunction with IL-3, cause myeloid leukemia. So why doesn't HOXB4 cause a hematological malignancy?

Our unexpected discovery may hold an answer. It is quite possible that some HSCs when stimulated with HOXB4 do become malignant. But as their phenotype changes, they undergo apoptosis. Tumors do not grow as fast as an embryo that typically grows to 3 kg in 9 months. Embryonic growth is explosive but organized. Strong regulatory mechanisms probably exist that prevent the development of malignancy during this period. HOXB4 may be such a mechanism in the hematopoietic system.

Our experiments demonstrate unexpectedly that HOXB4 has a therapeutic role in human B cell malignancies. Since HOXB4 is a natural protein, if it were to be given systemically, no antibody reaction would occur. The protein would enter various cell types by passive translocation, and in the case of HSCs, cause proliferation. B cell malignancies would undergo apoptosis. Other cells of the hematopoietic lineage would not be affected, for a period of therapy up to 1 year, as has been demonstrated in a mouse model.

But what would happen to the other 200-odd cell types in the body? Would they undergo proliferation, malignant transformation, apoptosis, dedifferentiation, or remain quiescent?

If the Aniridia paradigm is any guide, the results may be favorable. When the Aniridia gene is overexpressed in any part of the Drosophila body outside of the critical period, then there is no effect! There is no malignancy, no dedifferentiation, no proliferation, and no apoptosis. The gene is quite specific in its function. Indeed, HOXB4 overexpression in HSCs leads to successful bone marrow transplantation in mice. The HSCs and their progeny continue to express HOXB4, which by passive translocation must be disseminating throughout the body. No malignancy of any other system has been reported in such mice, indicating that at least low levels of HOXB4 are harmless to the rest of the body.

In HSCs, HOXB4 causes clonal expansion whether it is retrovirally expressed, enters the cell once it is fused with the HIV TAT protein, or passively translocates through the cell membrane. Therefore excess HOXB4, regardless of the method of production, has the same effect. In the notochord of Xenopus, HOXB4 protein can cause upregulation of FLASH leading to apoptosis.

Our findings provide substantive evidence that HOXB4 overexpression via lipofection and transmembrane delivery causes apoptosis in the REH, WSU-DLCL2, WSU-FSCCL, and WSU-WM cell lines. We also show that apoptosis via transmembrane delivery of HOXB4 protein is demonstrated using a technique that allows study of large cell numbers and permits Western blotting.

As one example only of such tests, a pBAD/HIS-HOXB4 plasmid was used to transform TOP10 E. coli bacteria. HOXB4 protein production was induced by L-arabinose. The protein was purified by Ni-NTA Magnetic Agarose Beads. Enterokinase (EK) was used to remove the histidine tag using the built-in EK digestion site. The pBAD/HIS-LACZ plasmid was used to produce β-galactosidase in a similar manner. REH, WSU-DLCL2, WSU-FSCCL, and WSU-WM cells were cultured with HOXB4 protein and β-galactosidase at concentrations varying from 0.05 μg/ml to 5 μg/ml. Caspase inhibitors and antisense oligonucleotide to FLASH was used to determine if the apoptosis can be abolished.

Immunocytochemistry would be performed to demonstrate intracellular HOXB4. Cell growth would be plotted with HOXB4 and β-galactosidase at different concentrations for each cell line to determine individual sensitivity. Western blots would be used to follow the time course of expression of FLASH, caspase-8, caspase-3, Bcl-2, Bax, XIAP, NAIP, cIAP-1, c-IAP-2, survivin, and poly(ADP-ribose) polymerase (PARP) cleavage. Activated caspases will also be measured by fluorescence microscopy. TUNEL assays would be performed. DNA laddering would be demonstrated by agarose gel electrophoresis. SMAC, and caspase-9 would also be measured to prove that the intrinsic pathway is not active. Cytochrome c release would quantified by sandwich ELISA.

Induction of apoptosis via transmembrane delivery of HOXB4 protein in all cell lines provides additional in vitro evidence for the potential efficacy of HOXB4 protein in B cell malignancies. Analysis of caspases activation and measurement of mediator proteins would distinguish between the intrinsic and extrinsic pathways of apoptosis. Presence of FLASH protein would confirm that this effect is mediated via caspase-8. Time course studies would elucidate this process further. Abrogation of apoptosis with FLASH and caspase-8 inhibition would prove that the effect of HOXB4 is largely, if not solely, mediated via FLASH and caspase-8.

Alternatively, a fusion protein, TAT-HOXB4, has been described that also passively translocates across cell membranes and enters HSCs using the property of HIV TAT protein to enter cells. Ecdysone-inducible expression of HOXB4 can be attempted to produce high levels of endogenous protein. Tetracycline-inducible systems have also been commonly used, and are another alternative. Glucocorticoid receptor fused with HOXB4 can be expressed via plasmid transfection. The glucocorticoid receptor binds to heat shock protein HSP90 that prevents nuclear entry. Dexamethasone releases the HSP90 leading to nuclear entry. Since corticosteroids are lymphotoxic and part of standard therapy for lymphoma, this model would not be our first choice. Other alternatives include (a) fusing HOXB4 with an immunoglobulin signal peptide that does not allow it to enter the nucleus but promotes secretion into the culture medium, and (b) co-culture with MS-5 mouse stromal cell line that constitutively expresses HOXB4.

Other non-hematological malignancies may also be susceptible to HOXB4. We have evidence that 293 cells (derived from kidney embryonal cells) also undergo apoptosis with HOXB4.

Our findings provide substantive evidence that HOXB4 administration induces apoptosis in SCID mice tumors derived from REH, WSU-DLCL2, WSU-FSCCL, and WSU-WM cells by injection of HOXB4 protein. Our studies provide evidence that HOXB4 induces apoptosis in WSU-DLCL2 tumors in SCID mice. The therapeutic potential may be expanded by extension to REH, WSU-FSCCL, and WSU-WM tumors in SCID mice.

Our unexpected findings show that HOXB4 induces apoptosis in REH, WSU-DLCL2, WSU-FSCCL, and WSU-WM cells in vitro. We have also provided in vivo evidence that WSU-DLCL2 tumors are reduced in size in SCID mice by injection of HOXB4 protein. The therapeutic potential may be expanded by inducing apoptosis in REH, WSU-DLCL2, WSU-FSCCL, and WSU-WM tumors in SCID mice.

As one example only, without limitation, tumors derived from REH, WSU-DLCL2, WSU-FSCCL, and WSU-WM cell lines would be created by tumor transplantation. Doses of HOXB4 protein varying from 2 μg to 100 μg would be injected directly into the tumors and surrounding tissue every day for 5 days. The animals would be euthanized and tumors would be harvested to study the apoptosis pathway.

The HOXB4 protein would also be injected intraperitoneally to examine if HOXB4 protein can act systemically to cause apoptosis in tumors.

Tumor growth would be plotted for each concentration tested to determine susceptibility to HOXB4. Protein would be extracted from harvested tumors. Western blots would be used to follow the time course of expression of FLASH, caspase-8, caspase-3, Bcl-2, Bax, XIAP, NAIP, cIAP-1, c-IAP-2, survivin, and poly(ADP-ribose) polymerase (PARP) cleavage. TUNEL assays would be performed. DNA laddering would be demonstrated by agarose gel electrophoresis. SMAC, and caspase-9 will also be measured to prove that the intrinsic pathway is not active. Cytochrome c release would be quantified by sandwich ELISA. Any adverse effects on the mice would be carefully recorded.

Reduction in tumor size in vivo for all cell lines would confirm the activity of HOXB4 across the entire spectrum of B cell malignancies. Induction of apoptosis via intraperitoneal injection would demonstrate the potential of systemic therapy with HOXB4. Release of cytochrome c and DNA laddering would confirm apoptosis. Analysis of caspases activation and measurement of mediator proteins would distinguish between the intrinsic and extrinsic pathways of apoptosis. Presence of FLASH protein would confirm that this effect is mediated via caspase-8. Time course studies would elucidate this process further.

### EXAMPLE 3

As some examples only, without limitation, the following research design and methods may be usable in evaluating and implementing some embodiments of the invention:

A. Cell Culture: REH cells and other cell lines would be routinely cultured in suspension using RPMI 1640 medium with L-glutamine and 10% heat-inactivated fetal calf serum at 37° C. and 5% CO₂ in 25 cm² flasks. Medium would be changed every 3 to 7 days, depending on growth rate. G418 would be used for selection of neomycin resistance. ER-CHO, ER-NIH3T3, and ER-293 cells may also be culture in DMEM if needed. Since co-culture experiments are planned, we will culture these cell lines in the richer RPMI medium when possible.

B. Plasmid Transfection: The pLNCX2-HOXB4, pERV2, pEGSH-Luc, and pEGSH-B4 (and control) plasmids would be transfected into REH cells using Lipofectamine Plus (Invitrogen, Carlsbad, Calif.) according to the manufacturer's protocol. Similar method would be used for ER-CHO, ER-NIH3T3, and ER-293 cells. Neomycin selective pressure would be applied by adding G418 to the culture after 3 days. Hygromycin would be used for pEGSH transfectants. Cells would be allowed to grow in the medium as above.

C. Immunocytochemistry: Using a Cytospin centrifuge (Thermo Electron, Waltham, Mass.), REH cells in suspension would be deposited on a glass slide. Cells would be fixed with 4% methanol free formaldehyde and then washed with PBS for 5 minutes. The slide would be incubated with the primary antibody for one hour at room temperature and then left at 4° C. overnight. The slide would be washed with PBS 3 times for 5 minutes each. The slide will then be incubated with the secondary biotinylated antibody for 3 hours at room temperature. After another wash cycle, the Vectastain ABC kit (Vector Labs, Burlingame, Calif.) would be used to stain the cells.

D. Western Blot: Protein would be extracted from REH cells using the CelLytic MEM Protein Extraction Kit (Sigma Aldrich, St Louis, Mo.). The extract would be run on SDS-PAGE and electrically transferred to nylon membrane. The membrane would be blotted with the appropriate antibody, and the Immun-Star-AP Chemiluminescent Kit (Bio-Rad, Hercules, Calif.) would be used for detection.

E. Microarray: Using the PCR primers we have already designed, human genomic DNA would be used to amplify all 80 genes of these 3 protein families. The product would be confirmed by size and sequence, and would be purified and concentrated using Wizard PCR Preps (Promega, Madison, Wis.). The Microarray Facility at Wayne State University will fabricate the microarray. RNA would be extracted from cells using the RNeasy Mini Kit (Qiagen, Valencia, Calif.). It would be labeled with Cy3 or Cy5 using the Atlas PowerScript Fluorescent Labeling Kit (BD Biosciences, Palo Alto, Calif.) and hybridized to the microarray using the Atlas Hybridization Chamber. Data would be acquired by a laser scanner at the Microarray Facility. GenePix Pro software (Axon, Union City, Calif.) would be used to subtract background, normalize to control genes, and calculate expression ratios using duplicates on the microarray slide. A two-fold ratio would be considered significant to explore further with “real-time” RT-PCR.

F. Ecdysone-inducible Expression: Using the Complete Control Inducible Mammalian Expression System (Stratagene, La Jolla, Calif.), the cells would be transfected with the pERV3 plasmid containing both ecdysone receptor subunits separated by an IRES, and then subsequently with the pEGSH-HOXB4 plasmid using the manufacturer's instructions. Cells would be grown on agar, and single colonies would be picked to ensure monoclonality. Protein expression would be induced with ecdysone-analog ponasterone A and cells would be checked for HOXB4 expression by immunocytochemistry, Western blotting, and by flow cytometry utilizing the co-expressed GFP.

G. TUNEL Assay: Cells would be attached to a glass microscope slide by Cytospin centrifuge (Thermo Electron, Waltham, Mass.). The slide would be immersed in 4% methanol-free formaldehyde for fixation. The DeadEnd Fluorometric TUNEL System (Promega, Madison, Wis.) would be used to label the cells. The slides would be visualized under a fluorescent microscope and photographed.

H. Caspase Activation: Caspase activation kits (Biocarta, San Diego, Calif.) would be used to detect activation of caspases 1, 2, 3, 4, 5, 6, 7, 8, and 9. The caspase inhibitors (included in the kit) would be used to determine if apoptosis can be abrogated or diminished.

I. Cytochrome c Release: Release of cytochrome c would be measured by Quantikine kit (R&D Systems, Minneapolis, Minn.) from cell lysates using a sandwich ELISA according to the manufacturer's instructions.

J. DNA Laddering: DNA would be extracted from REH cells using Wizard MiniPreps (Promega, Madison, Wis.) and separated by agarose gel electrophoresis to demonstrate laddering.

K. “Real-time” RT-PCR: Gene sequences of members of the Homeobox, Trithorax, and Polycomb families whose RNA levels that are found to be elevated or reduced in response to overexpression of HOXB4 would be examined. If any splice variants are known, the relevant portions of the native RNA would be excluded. Two different regions, preferably from the 5′ and 3′ end of the RNA would be examined. Using Primer3 software, PCR primers would be designed to amplify 50 to 100 base pair segments of the RNA. RNA would be extracted from REH cells. It would be reverse transcribed to cDNA and then amplified in a “real-time quantitative” PCR machine, LightCycler (Roche, Basel, Switzerland).

L. Protein Production and Purification: TOP10 E. coli (Invitrogen, Carlsbad, Calif.) would be transformed with pBAD/HIS-HOXB4 or pBAD/HIS-LACZ plasmid and selected on LB plates with ampicillin. HOXB4 protein production would be induced with 0.02% L-arabinose. Protein would be extracted from cell culture by using a lysis buffer with Tween. Lysozyme would be added and cells sonicated and centrifuged. Supernatant containing the histidine-tagged protein would be purified using Ni-NTA Magnetic Agarose Beads (QIAGEN, Valencia, Calif.) under native conditions using the manufacturer's protocol. The purified protein would be digested with enterokinase to remove the histidine tag, and the histidine tag would be removed from solution by binding to the nickel column.

M. Tumor Induction in SCID Mice: To initiate the xenografts, 10⁷ cells would be injected subcutaneously and bilaterally in the flank areas of two SCID mice. Animals would be observed for development of subcutaneous tumors at the sites of injection. When tumor size reaches 2000 mg, animals would be euthanized and tumors removed and dissected into small pieces (˜30 mg). A portion of the tumor would be transplanted subcutaneously into flanks of a new group of SCID mice using a 12-gauge trocar. To avoid discomfort and stress, animals would be euthanized when their total tumor burden reaches 2 g (10% body weight). All studies involving mice should be approved by the Institutional Animal Care and Use Committee (IACUC), which is known as the Animal Investigation Committee (AIC) at Wayne State University.

N. Tumor Injections: Since the tumors are small, only 25 μl can be safely injected into them. Depending on the dose and concentration of HOXB4, any remaining injectate would be delivered to the surrounding subcutaneous tissue. Intraperitoneal injections would be given in a standard manner.

### EXAMPLE 4

Pathogenesis of primary pulmonary mucosa-associated lymphoid tumors (MALT) and lymphomas remains unknown, but it is thought to involve antigen stimulation leading to immortalization, and then subsequent transformation into a more aggressive phenotype. As noted herein, we have shown unexpectedly that HOXB4 can induce apoptosis in certain malignant cell lines through a caspase-mediated pathway involving FLASH and caspase-8, with caspase-3 as the effector caspase. We also show herein that HOXB4 induces apoptosis through an alternate novel pathway.

HOXB4 protein was purified using a Ni-NTA purification system and confirmed with a Western blot. HOXB4 protein was added, at varying concentrations up to 1 μg/ml, to cell cultures of several malignant cell lines including WSU-DLCL2, WSU-FSCCL and REH. Initial growth curve were obtained demonstrating cell death with the addition of HOXB4. Western blots demonstrated increased expression of FLASH, caspase-3, and caspase-8. Cell lines treated with HOXB4 were then individually treated with inhibitors to each of the caspases. Subsequent growth curves were plotted, and Western blots obtained. Cells treated with the caspase-3 inhibitor exhibited cell growth. However, cells treated with caspase-8 inhibitor still underwent apoptosis.

Our results show that HOXB4 induced apoptosis has previously been shown to increase the expression of FLASH, caspase-3, and caspase-8, implicating a possible role of caspase-8 in apoptosis. Our data shows that cells treated with caspase-8 inhibitor fail to demonstrate an arrest of apoptosis.

HOXB4 induced apoptosis has been previously implicated through a pathway consisting of FLASH, caspase-8 and caspase-3. The failure of apoptosis to be completely abolished by a caspase-8 inhibitor indicates a role for a second pathway. We hypothesize that FLASH may activate a second pathway via activation of caspase-10, with subsequent activation of caspase-3. Alternatively in Drosophila, the homolog of HOXB4 called, Deformed, activates the homolog of SMAC, called Reaper. SMAC, a second mitochondria-derived activator or caspase has been implicated to function through a pathway that involves caspase-9 in humans. This is presently under investigation in our laboratory.

Our results suggest that HOXB4 induced apoptosis may occur through a multifactorial pathway. This provides a potential therapeutic advantage against rapidly dividing malignant cells.

As discussed previously, HOXB4 is a homeodomain transcription factor with diverse roles in embryonic development and the regulation of stem cells. HOXB4 induces apoptosis in embryonic structures while promoting the development and proliferation of certain stem cell lines. When HOXB4 is overexpressed in embryonic stem cells, differentiation into hematopoietic stem cells (“HSCs”) occurs. Moreover, when HOXB4 is highly overexpressed in hematopoietic stem cells (HSCs) there is clonal expansion of HSCs preventing their differentiation into more terminal cell lines. Lower levels of HOXB4 expression allow myeloid differentiation of HSCs.

We have previously demonstrated that HOXB4 has a second role, that is, it causes apoptosis in the REH pre-B ALL cell line by passive translocation across the cell membrane. Our work suggests that the apoptotic effect of HOXB4 via transmembrane delivery may not be entirely via the previously proposed caspase-8 mechanism.

HOXB4 cDNA was ligated into the bacterial expression vector pBAD in frame with the 6-histidine tag and used to transform the TOP 10 E. coli bacterial strain. Protein production was induced with L-arabinose. HOXB4 protein was purified using a Ni-NTA purification system (QIAGEN, Valencia, Calif.) and confirmed with a Western blot using an antibody to the tagged Xpress epitope and a HOXB4 rat monoclonal antibody (Developmental Studies Hybridoma Bank, University of Iowa).

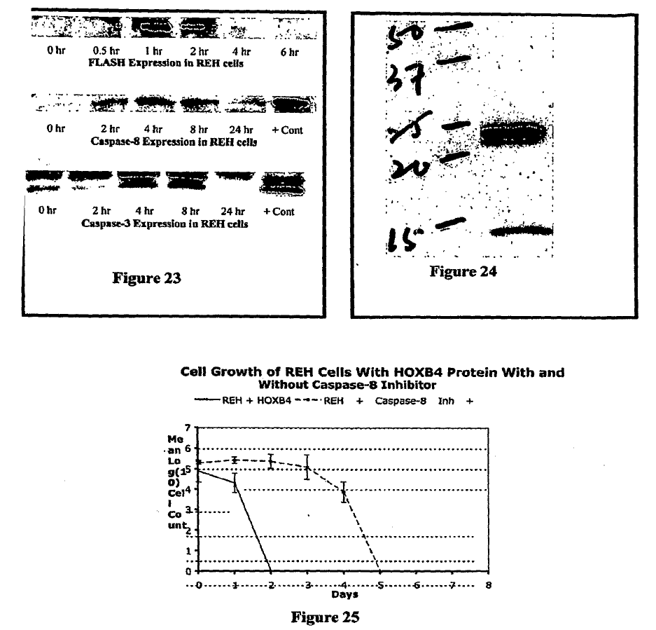

The REH cell lines were cultured with 1 μg/ml of HOXB4. Cell protein was extracted at various time points by lysis. Western blots were performed with antibodies to caspase-3 (Santa Cruz Biotechnology, Santa Cruz, Calif.), caspase-8 (Leinco, St Louis, Mo.), and FLASH (Imgenex, San Diego, Calif.), also called caspase-8 associated protein-2. A time course of expression is shown (FIG. 23).

A caspase-8 inhibitor, Z-IETD-FMK, was obtained from the manufacturer (Biocompare, Mountain View, Calif.) and added to a culture of REH cells in RPMI medium. HOXB4 was then added to induce apoptosis.

Our results show a Western blot demonstrates a protein of the predicted size—25 kD (FIG. 24). A smaller protein of about 15 kD is also seen. This probably represents a breakdown product of the native protein.

Western blots of REH cells undergoing apoptosis indicate increased expression of caspase-3, caspase-8, and FLASH (Casp8ap2) confirming the process of apoptosis.

Addition of the cell-permeable caspase-8 inhibitor leads to delay in cell death, but does not lead to cell survival. All REH cells exposed to HOXB4 eventually die.

We have shown before that HOXB4 induces apoptosis in REH cells via the expression of FLASH, caspase-8, and caspase-3 (e.g., FIG. 8). This mechanism is similar that seen in the notochord of Xenopus. However, in Drosophila the homolog of HOXB4, called Deformed, causes apoptosis in the head of the developing fly by activation of the gene Reaper. This mechanism is distinct from the Xenopus mechanism.

We are unable to completely inhibit the process of apoptosis by inhibition of caspase-8 (e.g., FIG. 25). Therefore it is possible that other mediators of apoptosis are also involved. FLASH can activate caspase-10, leading to activation of caspase-3. The human homolog of Reaper is SMAC, second mitochondrial activator of caspase. Activation of this protein by HOXB4 can also lead to apoptosis

HOXB4-induced apoptosis of REH cells may not be a phenomenon with a single mechanism. Multiple pathways may be involved. Elucidation of these pathways requires further study.

Therapy with HOXB4 protein may provide benefits in hematological malignancies, such that cancer cells may undergo apoptosis without concomitant systemic immunosuppression. This will occur due to the ability of HOXB4 to deliver enhancement of immunity through expansion of HSCs. Since HOXB4 may be inducing apoptosis via multiple mechanisms, this approach to therapy may be a robust one. Cancer cells may find it difficult to evade a plurality of apoptotic inducers, and therefore remain susceptible to HOXB4 therapy.

### EXAMPLE 5

As discussed herein, we have demonstrated unexpectedly HOXB4 induces apoptosis in multiple B-cell lines derived from hematological malignancies. These cell lines include REH, WSU-DLCL2, WSU-FSCCL, and WSU-WM, representing the entire differentiation spectrum of B cell malignancies (REH being least mature, to WSU-WM most mature). It has been shown by other investigators, that HOXB4 causes clonal expansion of hematopoietic stem cells. Demonstration of both of these effects relies upon the unusual property of homeobox proteins to passively cross cell membranes (e.g. without receptor mediation, pinocytosis, etc.) due to the triple-helix structure of the homeodomain. Taken together, these two properties indicate that we have discovered a natural protein that is able to differentiate malignant cells from normal cells and selectively eliminate malignancy.

In accordance with some embodiments of the invention, those of ordinary skill will appreciate methods, compositions, and systems to investigate the stability and pharmacokinetics of HOXB4 protein.

As some examples only, without limitation, both human and mouse HOXB4 proteins would be synthesized via bacterial production using TOP10 E. coli and pBAD plasmid incorporating a 6-histidine tag. After nickel column purification, the histidine tag would be removed by cleavage at the built-in enterokinase digestion site. The purified protein would be tested against REH cells to examine its activity and stability compared to the nickel column eluate and control protein (β-galactosidase). Biological stability would be evaluated by incubating the protein at 4° C., 25° C., and 37° C. for periods up to 24 hours and constructing dose-response curves.

Both mouse and human proteins would be injected into mice intravenously, subcutaneously, and intraperitoneally. Blood would be drawn at 5 minutes, 10 minutes, 15 minutes, 30 minutes, 1 hour, 4 hours, 8 hours, and 24 hours. Protein would be assayed by radioimmunoassay (RIA) and confirmed by ELISA using a monoclonal antibody to HOXB4. A dose and route of HOXB4 would be chosen that mimics the concentration curve of the daily addition of 1 μg/ml of HOXB4 to RPMI culture medium. (This dose achieves complete cell death of all cell lines tested within 72-96 hours.)

In similar fashion, in accordance with some embodiments of the invention, those of ordinary skill will appreciate methods, compositions, and systems to investigate in vivo efficacy of HOXB4. For example, a simple tumor model would be created using DLCL2 and WSU-WM cells. Briefly, 10 million tumor cells would be injected subcutaneously into SCID mice and allowed to form tumors. Tumors would be harvested and 2-3 mg of tumor would be transplanted into SCID mice to propagate the tumors.

Our findings show tumor shrinkage in this model. Moreover, using results of the above technique, HOXB4 might be administered to SCID mice for an exemplary period up to one month. Tumor shrinkage would be measured measuring size and converting to milligrams using our previous experience. Tumors would be harvested at weekly intervals, and expression of FLASH/Diablo, caspase-8, and caspase-3 would be assayed by immunohistochemistry. Sacrificed mice would be examined for any gross abnormalities on necropsy. Blood would be tested for routine hematological and biochemical parameters to ensure no toxicity has developed.

In similar fashion, in accordance with some embodiments of the invention, those of ordinary skill will appreciate methods, compositions, and systems to investigate the differential effects of HOXB4 on cancer cells and normal stem cells. As one example only, bone marrow specimens from patients with ALL would be used and CD34-positive cells would be separated by flow cytometry. The cells would be cultured in RPMI 1640 medium with and without HOXB4 protein and cell survival examined. Normal hematopoietic stem cells would be obtained from bone marrow specimens of patients with normal findings on bone marrow examination and processed in a similar manner.

Dose-response curve of HOXB4 with cell growth and death would be constructed for normal and malignant hematopoietic stem cells. Immunohistochemistry would be performed on apoptotic cells to demonstrate activation of FLASH/Diablo, caspase-8, and caspase-3. DNA disintegration would be determined by a TUNEL assay.

In similar fashion, in accordance with some embodiments of the invention, those of ordinary skill will appreciate methods, compositions, and systems to further investigate and implement HOXB4 therapy in human leukemia. As one example only, an advanced leukemia model with greater clinical relevance would be created using bone marrow from patients with hematological malignancies. Seven-week old NOD/SCID mice would be irradiated (2.2 Gy) with a cesium-137 source. After 4 to 6 hours, mice would be inoculated by tail vein injection with 2 million to 10 million primary ALL specimens in RPMI 1640. After 1 month, mice would be monitored at 2-week intervals for human ALL engraftment by collecting 50 μl of blood from the retro-orbital sinus for flow cytometry analysis.

When CD45-positive cells reach 1-5%, 5-10%, 10-25%, and 25-50%, mice would be treated with HOXB4 protein based on dose and route as previously determined. After 4 weeks of treatment, 50 μl of blood would be drawn again to determine the CD45-positivity at weekly intervals. Reduction of CD45-positivity by 50% or more would be considered successful therapy. If complete clearance of CD45-positive cells is achieved, time to recurrence would be used as a measure of efficacy. Dose-response curves would be constructed.

Sacrificed mice would be examined for any gross abnormalities on necropsy. Spleens and other organs would be fixed in 10% neutral-buffered formalin for 24 hours, transferred to 70% ethanol before being embedded in paraffin and sectioned. Femurs would be fixed in formalin, then decalcified for 48 hours in 0.38 M disodium EDTA before being transferred to 70% ethanol, embedded in paraffin, and sectioned. Blood would be tested for routine hematological and biochemical parameters to ensure no toxicity has developed.

Cell suspensions of spleens and other organs would be prepared by mincing the tissues and filtering through 70-m cell strainers. Bone marrow would be collected by flushing femurs with RPMI 1640 containing 10% FBS. Mononuclear cells would be purified by density gradient centrifugation, and cells would be cryopreserved in FBS with 10% DMSO. The proportions of human versus mouse CD45 cells in bone marrow, peripheral blood, and minced tissues will quantified by flow cytometry to determine the level of engraftment. Reduction in engraftment by 50% or more would be considered successful therapy. If complete clearance of CD45-positive cells is achieved, time to recurrence would be used as a measure of efficacy.

All cited references are hereby incorporated by reference in full as though fully set forth herein.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements. It is intended that the following claims define the scope of the invention and that the systems, methods, and compositions within the scope of these claims and their equivalents be covered thereby.

| SEQ ID NO. 1, Human HOXB4 Protein Sequence: | |
|---|---|
| 1 | mamssflins nyvdpkfppc eeysqsdylp sdhspgyyag gqrressfqp eagfgrraac |
| 61 | tvqryaacrd pgpppppppp pppppppgls prapapppag allpepgqrc eavssspppp |
| 121 | pcaqnplhps pshsackepv vypwmrkvhv stvnpnyagg epkrsrtayt rqqvleleke |
| 181 | fhynryltrr rrveiahalc lserqikiwf qnrrmkwkkd hklpntkirs ggaagsaggp |
| 241 | pgrpnggpra l |
| Can be found at: | |
| http://www.ncbi.nlm.nih.gov/entrez/viewer.fcgi?db = protein&val = | |
| 12007115 | |
| SEQ ID NO.2, Human HOXB4 Nucleotide Sequence: | |
| 1 | atggctatga gttctttttt gatcaactca aactatgtcg accccaagtt ccctccatgc |
| 61 | gaggaatatt cacagagcga ttacctaccc agcgaccact cgcccgggta ctacgccggc |
| 121 | ggccagaggc gagagagcag cttccagccg gaggcgggct tcgggcggcg cgcggcgtgc |
| 181 | accgtgcagc gctacgcggc ctgccgggac cctgggcccc cgccgcctcc gccaccaccc |
| 241 | ccgccgcccc cgccaccgcc cggtctgtcc cctcgggctc ctgcgccgcc acccgccggg |
| 301 | gccctcctcc cggagcccgg ccagcgctgc gaggcggtca gcagcagccc cccgccgcct |
| 361 | ccctgcgccc agaaccccct gcaccccagc ccgtcccact ccgcgtgcaa agagcccgtc |
| 421 | gtctacccct ggatgcgcaa agttcacgtg agcacggtaa accccaatta cgccggcggg |
| 481 | gagcccaagc gctctcggac cgcctacacg cgccagcagg tcttggagct ggagaaggaa |
| 541 | tttcactaca accgctacct gacacggcgc cggagggtgg agatcgccca cgcgctctgc |
| 601 | ctctccgagc gccagatcaa gatctggttc cagaaccggc gcatgaagtg gaaaaaagac |
| 661 | cacaagttgc ccaacaccaa gatccgctcg ggtggtgcgg caggctcagc cggagggccc |
| 721 | cctggccggc ccaatggagg cccccgcgcg ctctag |

Can be found at:
http://www.ncbi.nlm.nih.gov/entrez/viewer.fcgi?val = 12007114&itemID =
1&view = gbwithparts

What is claimed is:

1. A method of decreasing the survival of cancer cells in a mammal, comprising the step of administering to the mammal a compound comprising HOXB4 protein in a therapeutically effective amount that decreases the survival of at least some of the cancer cells.

2. The method of claim 1, wherein the mammal is a human.

3. The method of claim 1 or claim 2, wherein the HOXB4 protein comprises a polypeptide of all or part of SEQ ID NO. 1.

4. A method of inducing apoptosis in cancer cells in a mammal, comprising the step of administering to the mammal a compound comprising HOXB4 protein in a therapeutically effective amount that induces apoptosis in at least some of the cancer cells.

5. The method of claim 4, wherein the mammal is a human.

6. The method of claim 4 or claim 5, wherein the HOXB4 protein comprises a polypeptide of all or part of SEQ ID NO. 1.

7. A method of decreasing the survival of cancer cells in a mammal, comprising the step of transfecting the mammal with a HOXB4 nucleotide sequence in a therapeutically effective amount that decreases the survival of at least some of the cancer cells.

8. The method of claim 7, wherein the mammal is a human.

9. The method of claim 7 or claim 8, wherein the HOXB4 nucleotide sequence comprises all or part of SEQ ID NO. 2.

10. A method of inducing apoptosis in cancer cells in a mammal, comprising the step of transfecting the mammal with a HOXB4 nucleotide sequence in a therapeutically effective amount that induces apoptosis in at least some of the cancer cells.

11. The method of claim 11, wherein the mammal is a human.

12. The method of claim 11 or claim 12, wherein the HOXB4 nucleotide sequence comprises all or part of SEQ ID NO. 2.

13. Use in a medicament of a compound comprising HOXB4 protein to decrease survival of cancer cells in a mammal.

14. The use of claim 13, wherein the mammal is a human.

15. The use of claim 13 or claim 14, wherein the HOXB4 protein comprises a polypeptide of all or part of SEQ ID NO. 1

16. Use in a medicament of a transfection agent comprising a HOXB4 nucleotide sequence to decrease survival of cancer cells in a mammal.

17. The use of claim 16, wherein the mammal is a human.

18. The use of claim 17 or claim 18, wherein the HOXB4 nucleotide sequence comprises a nucleotide sequence of all or part of SEQ ID NO. 2.

19. A pharmaceutical composition for treatment of cancer comprising a polypeptide of all or part of SEQ ID NO. 1 and a pharmaceutically acceptable carrier.

20. A pharmaceutical composition for treatment of cancer comprising a nucleotide sequence of all or part of SEQ ID NO. 2 and a pharmaceutically acceptable carrier.

21. A method of treating a mammalian patient having a malignancy, said method comprising administering to the patient a pharmaceutical composition according to claim 19 or claim 20 in a therapeutically effective amount.

22. The method according to claim 21, wherein the pharmaceutical composition is administered locally to a tumor site.

23. The method according to claim 21, wherein the pharmaceutical composition is administered by direct injection to a tumor.

24. The method according to claim 21, wherein the pharmaceutical composition is administered by intravenous injection.

25. The method according to claim 21, wherein the administration is performed at two or more separate times.
